SEQUENCE LISTING (42 sequences)

SEQ ID NO: 1 - RNA, 90 nt, Artificial Sequence
  Synthetic constructs
aacacaucagauuuccugguguaacgaauuuuuuaagugcuucuugcuuaagcaaguuucaucccgacccccucagggucgggauuuuuu

SEQ ID NO: 2 - RNA, 97 nt, Artificial Sequence
  Synthetic constructs
aacguuaagugaacauuccuccacuuaacaacgaauuuuuuaagugcuucuugcuuaagcaaguuucaucccgacccccucagggucgggauuuuuu

SEQ ID NO: 3 - RNA, 96 nt, Artificial Sequence
  Synthetic constructs
aacaagugagguuaacauuccuccacuuaacgaauuuuuuaagugcuucuugcuuaagcaaguuucaucccgacccccucagggucgggauuuuuu

SEQ ID NO: 4 - RNA, 98 nt, Artificial Sequence
  Synthetic constructs
aacuggagaaugcauguuaacauuccuccaaacgaauuuuuuaagugcuucuugcuuaagcaaguuucaucccgacccccucagggucgggauuuuuu

SEQ ID NO: 5 - RNA, 97 nt, Artificial Sequence
  Synthetic constructs
aacagaauguuauacauguuaacauuccuaacgaauuuuuuaagugcuucuugcuuaagcaaguuucaucccgacccccucagggucgggauuuuuu

SEQ ID NO: 6 - RNA, 98 nt, Artificial Sequence
  Synthetic constructs
aacaauguuaacucuauacauguuaacauuaacgaauuuuuuaagugcuucuugcuuaagcaaguuucaucccgacccccucagggucgggauuuuuu

SEQ ID NO: 7 - RNA, 97 nt, Artificial Sequence
  Synthetic constructs
aacguuaacauuaaucuauacauguuaacaacgaauuuuuuaagugcuucuugcuuaagcaaguuucaucccgacccccucagggucgggauuuuuu

SEQ ID NO: 8 - RNA, 96 nt, Artificial Sequence
  Synthetic constructs
aacauauguauaguaaucuauacauguuaacgaauuuuuuaagugcuucuugcuuaagcaaguuucaucccgacccccucagggucgggauuuuuu

SEQ ID NO: 9 - RNA, 97 nt, Artificial Sequence
  Synthetic constructs
aacauguauaguauuaguaaucuauacauaacgaauuuuuuaagugcuucuugcuuaagcaaguuucaucccgacccccucagggucgggauuuuuu

SEQ ID NO: 10 - RNA, 92 nt, Artificial Sequence
  Synthetic constructs
aacacaucagauuuccugguguaacgaauuuuuuaacauuccuccacuuaacgagaauguucaucccgacccccucagggucgggauuuuuu

SEQ ID NO: 11 - RNA, 92 nt, Artificial Sequence
  Synthetic constructs
aacacaucagauuuccugguguaacgaauuuuuuguuaacauuccuccacuuaauguuaaucaucccgacccccucagggucgggauuuuuu

SEQ ID NO: 12 - RNA, 92 nt, Artificial Sequence
  Synthetic constructs
aacacaucagauuuccugguguaacgaauuuuuucauguuaacauuccuccauguaacaugcaucccgacccccucagggucgggauuuuuu

SEQ ID NO: 13 - RNA, 91 nt, Artificial Sequence
  Synthetic constructs
aacacaucagauuuccugguguaacgaauuuuuuauacauguuaacauuccuacauguaucaucccgacccccucagggucgggauuuuuu

SEQ ID NO: 14 - RNA, 92 nt, Artificial Sequence
  Synthetic constructs
aacacaucagauuuccugguguaacgaauuuuuuucuauacauguuaacauucauguaagacaucccgacccccucagggucgggauuuuuu

SEQ ID NO: 15 - RNA, 92 nt, Artificial Sequence
  Synthetic constructs
aacacaucagauuuccugguguaacgaauuuuuuuaaucuauacauguuaacguaagauuacaucccgacccccucagggucgggauuuuuu

SEQ ID NO: 16 - RNA, 91 nt, Artificial Sequence
  Synthetic constructs
aacacaucagauuuccugguguaacgaauuuuuuuaguaaucuauacauguuagauacuacaucccgacccccucagggucgggauuuuuu

SEQ ID NO: 17 - RNA, 93 nt, Artificial Sequence
  Synthetic constructs
aacacaucagauuuccugguguaacgaauuuuuuuauuaguaaucuauacauauuauuaauacaucccgacccccucagggucgggauuuuuu

SEQ ID NO: 18 - RNA, 93 nt, Artificial Sequence
  Synthetic constructs
aacacaucagauuuccugguguaacgaauuuuuuaguuuauccucccaaaaugguauaaauucaucccgacccccucagggucgggauuuuuu

SEQ ID NO: 19 - RNA, 93 nt, Artificial Sequence
  Synthetic constructs
aacacaucagauuuccugguguaacgaauuuuuuaauguuuauccucccaaaaauaaauauucaucccgacccccucagggucgggauuuuuu

SEQ ID NO: 20 - RNA, 93 nt, Artificial Sequence
  Synthetic constructs
aacacaucagauuuccugguguaacgaauuuuuuaucauguuuauccucccaaaaacauuaucaucccgacccccucagggucgggauuuuuu

SEQ ID NO: 21 - RNA, 93 nt, Artificial Sequence
  Synthetic constructs
aacacaucagauuuccugguguaacgaauuuuuuaucauguuuauccucccaaaaacaugaucaucccgacccccucagggucgggauuuuuu

SEQ ID NO: 22 - RNA, 93 nt, Artificial Sequence
  Synthetic constructs
aacacaucagauuuccugguguaacgaauuuuuuauuucauguuuauccucccacauuaaaucaucccgacccccucagggucgggauuuuuu

SEQ ID NO: 23 - RNA, 93 nt, Artificial Sequence
  Synthetic constructs
aacacaucagauuuccugguguaacgaauuuuuuauuucauguuuauccucccacaugaaaucaucccgacccccucagggucgggauuuuuu

SEQ ID NO: 24 - RNA, 93 nt, Artificial Sequence
  Synthetic constructs
aacacaucagauuuccugguguaacgaauuuuuuaguuuucauguuuauccucaugaaaauucaucccgacccccucagggucgggauuuuuu

SEQ ID NO: 25 - RNA, 93 nt, Artificial Sequence
  Synthetic constructs
aacacaucagauuuccugguguaacgaauuuuuuauuguuuucauguuuauccgaaaauaaucaucccgacccccucagggucgggauuuuuu

SEQ ID NO: 26 - RNA, 93 nt, Artificial Sequence
  Synthetic constructs
aacacaucagauuuccugguguaacgaauuuuuuauauuguuuucauguuuauaaacaauaucaucccgacccccucagggucgggauuuuuu

SEQ ID NO: 27 - RNA, 93 nt, Artificial Sequence
  Synthetic constructs
aacacaucagauuuccugguguaacgaauuuuuuaauuauuguuuucauguuuacaauaauucaucccgacccccucagggucgggauuuuuu

SEQ ID NO: 28 - RNA, 93 nt, Artificial Sequence
  Synthetic constructs
aacacaucagauuuccugguguaacgaauuuuuuaagauuauuguuuucauguaauaauuuucaucccgacccccucagggucgggauuuuuu

SEQ ID NO: 29 - RNA, 93 nt, Artificial Sequence
  Synthetic constructs
aacacaucagauuuccugguguaacgaauuuuuuauaagauuauuguuuucauuaaucuuaucaucccgacccccucagggucgggauuuuuu

SEQ ID NO: 30 - RNA, 101 nt, Artificial Sequence
  Synthetic constructs
aacauugggaggauucauguuuauccucccaauaacgaauuuuuuaagugcuucuugcuuaagcaaguuucaucccgacccccucagggucgggauuuuuu

SEQ ID NO: 31 - RNA, 101 nt, Artificial Sequence
  Synthetic constructs
aacauugugaggauucauguuuauccucccaauaacgaauuuuuuaagugcuucuugcuuaagcaaguuucaucccgacccccucagggucgggauuuuuu

SEQ ID NO: 32 - RNA, 101 nt, Artificial Sequence
  Synthetic constructs
aacagggaggauaauuucauguuuauccucccuaacgaauuuuuuaagugcuucuugcuuaagcaaguuucaucccgacccccucagggucgggauuuuuu

SEQ ID NO: 33 - RNA, 101 nt, Artificial Sequence
  Synthetic constructs
aacagugaggauaauuucauguuuauccucccuaacgaauuuuuuaagugcuucuugcuuaagcaaguuucaucccgacccccucagggucgggauuuuuu

SEQ ID NO: 34 - RNA, 100 nt, Artificial Sequence
  Synthetic constructs
aacguuaagugaacauuccuccacuuaacaacgaauuuuuuaucauguuuauccucccaaaaacauuaucaucccgacccccucagggucgggauuuuuu

SEQ ID NO: 35 - RNA, 100 nt, Artificial Sequence
  Synthetic constructs
aacguuaagugaacauuccuccacuuaacaacgaauuuuuuauuucauguuuauccucccacaugaaaucaucccgacccccucagggucgggauuuuuu

SEQ ID NO: 36 - RNA, 100 nt, Artificial Sequence
  Synthetic constructs
aacguuaagugaacauuccuccacuuaacaacgaauuuuuuauauuguuuucauguuuauaaacaauaucaucccgacccccucagggucgggauuuuuu

SEQ ID NO: 37 - RNA, 100 nt, Artificial Sequence
  Synthetic constructs
aacagaauguuauacauguuaacauuccuaacgaauuuuuuaucauguuuauccucccaaaaacauuaucaucccgacccccucagggucgggauuuuuu

SEQ ID NO: 38 - RNA, 100 nt, Artificial Sequence
  Synthetic constructs
aacagaauguuauacauguuaacauuccuaacgaauuuuuuauuucauguuuauccucccacaugaaaucaucccgacccccucagggucgggauuuuuu

SEQ ID NO: 39 - RNA, 100 nt, Artificial Sequence
  Synthetic constructs
aacagaauguuauacauguuaacauuccuaacgaauuuuuuauauuguuuucauguuuauaaacaauaucaucccgacccccucagggucgggauuuuuu

SEQ ID NO: 40 - RNA, 100 nt, Artificial Sequence
  Synthetic constructs
aacguuaacauuaaucuauacauguuaacaacgaauuuuuuaucauguuuauccucccaaaaacauuaucaucccgacccccucagggucgggauuuuuu

SEQ ID NO: 41 - RNA, 100 nt, Artificial Sequence
  Synthetic constructs
aacguuaacauuaaucuauacauguuaacaacgaauuuuuuauuucauguuuauccucccacaugaaaucaucccgacccccucagggucgggauuuuuu

SEQ ID NO: 42 - RNA, 100 nt, Artificial Sequence
  Synthetic constructs
aacguuaacauuaaucuauacauguuaacaacgaauuuuuuauauuguuuucauguuuauaaacaauaucaucccgacccccucagggucgggauuuuuu

(19) United States
(12) Patent Application Publication
(10) Pub. No.: US 2018/0223283 A1
(43) Pub. Date: Aug. 9, 2018

(54) ANTISENSE FINGERLOOP RNAS AND USES THEREOF

(71) Applicant: Ohio State Innovation Foundation, Columbus, OH (US)

(72) Inventors: Richard A. Lease, Westerville, OH (US); Ashwin Lahiry, Columbus, OH (US); Samuel D. Stimple, Columbus, OH (US)

(21) Appl. No.: 15/865,983

(22) Filed: Jan. 9, 2018

Related U.S. Application Data
(60) Provisional application No. 62/454,404, filed on Feb. 3, 2017.
(60) Provisional application No. 62/533,857, filed on Jul. 18, 2017.
(60) Provisional application No. 62/562,105, filed on Sep. 22, 2017.

(51) Int. Cl. C12N 15/113; C12Q 1/6876

(52) U.S. Cl. CPC C12N 15/113; C12N 2310/531; C12Q 1/6876

(57) ABSTRACT

The present disclosure relates to chimeric antisense nucleotides and methods for modulating protein expression levels and/or RNA stability from at least two target mRNAs in a cell simultaneously.

### CROSS REFERENCE TO RELATED APPLICATIONS

This application claims the benefit of U.S. Provisional Patent Application Ser. No. 62/454,404 filed Feb. 3, 2017, U.S. Provisional Patent Application Ser. No. 62/533,857 filed Jul. 18, 2017, and U.S. Provisional Patent Application Ser. No. 62/562,105 filed Sep. 22, 2017, each of which are expressly incorporated herein by reference.

### STATEMENT REGARDING FEDERALLY SPONSORED RESEARCH

This invention was made with Government Support under Grant No. 1158394 awarded by the National Science Foundation. The Government has certain rights in the invention.

### REFERENCE TO SEQUENCE LISTING

The Sequence Listing submitted Jan. 9, 2018, as a text file named “10336-296US1_2018_01_09 Sequence_Listing_ST25.txt,” created on Jan. 8, 2018, and having a size of 11,590 bytes, is hereby incorporated by reference pursuant to 37 C.F.R. § 1.52(e)(5).

### FIELD

The present disclosure relates to chimeric antisense nucleotides and methods for modulating protein expression levels and/or RNA stability from at least two target mRNAs in a cell simultaneously.

### BACKGROUND

The use of RNA in synthetic biology applications has enormous potential. RNAs are programmable, tunable, and modular molecular tools with applications in metabolic and genome engineering, as well as in environmental, therapeutic, diagnostic and biotechnological fields. RNA tools have been successfully developed for altering gene expression, building genetic circuitry, and for sensing small molecules and other environmental cues.

Small non-coding RNAs, such as miRNAs, siRNAs and piRNAs, all about 21-32 nt in length, are one of the main and crucial classes of posttranscriptional gene regulators in eukaryotes. Being expressed under tight spatial and temporal control, small non-coding RNAs influence all aspects of organism's biology, including its development, metabolism and response to environmental conditions.

The importance of another class of small non-coding RNAs, bacterial small regulatory RNAs (sRNAs), became apparent only recently due to ongoing intensive research in prokaryote genetics and genomics. The bacterial small regulatory RNAs (sRNAs) are short RNA sequences (˜50-300 nucleotides) that alter the expression of protein-coding messenger RNAs (mRNAs) by RNA:RNA base-pairing interactions. sRNAs are a phenomenon of essentially all bacteria and represent a fundamental basis of genetic regulation. Many sRNAs govern gene expression at the level of mRNA translation control and are frequently found as stress response regulators.

Compared to conventional metabolic engineering approaches such as gene knockouts, the sRNAs of bacteria present the distinct advantage of being able to “tune” gene expression, modulating mRNA translation levels with relatively fine control. However, while single sRNAs have been examined for tuning gene expression, systems and methods that allow targeting multiple mRNAs and multiple genes simultaneously are needed. Multi-acting sRNAs targeting multiple target mRNAs could be especially useful tools for metabolic engineering because multiple mRNAs can be targeted by a single sRNA for simultaneous and coordinated translational regulation at different points in a metabolic pathway.

The systems and methods disclosed herein address these and other needs.

### SUMMARY

The inventors have developed novel systems and methods to measure the activity of chimeric nucleic acids in a cell and methods of measuring and modulating protein expression levels and/or RNA stability from multiple mRNAs in a cell simultaneously. The inventors have retargeted the small regulatory RNA DsrA to act at two or more alternative mRNA transcripts by retargeting the stem-loop antisense motifs. The inventors have developed the stem-loop antisense motifs of DsrA into a novel modular, general purpose RNA antisense-encoding structure. In some embodiments, transcription of each gene (one chimeric nucleic acid and two mRNAs) is separately and orthogonally controlled with small molecule inducers.

In one aspect, disclosed herein is a system for measuring the activity of a chimeric nucleic acid in a cell, comprising:
a first plasmid comprising a chimeric nucleic acid, wherein the chimeric nucleic acid comprises a first nucleic acid sequence operably linked to a second nucleic acid sequence;
a second plasmid comprising a first reporter gene operably linked to a first gene leader sequence; and
a third plasmid comprising a second reporter gene operably linked to a second gene leader sequence;
wherein the first nucleic acid sequence is present in a first fingerloop stem loop and the second nucleic acid sequence is present in a second fingerloop stem loop; and
wherein the first and second fingerloop stem loops inhibit the binding of the first and second nucleic acids to mismatched target sequences.

In some embodiments, the chimeric nucleic acid encodes an sRNA comprising a fingerloop structure. In some embodiments, the first nucleic acid sequence and the second nucleic acid sequence encode an sRNA comprised in stem-loop antisense structures. In some embodiments, the first nucleic acid sequence and the second nucleic acid sequence encode an sRNA comprising at least two fingerloop structures. In some embodiments, the first nucleic acid sequence and the second nucleic acid sequence encode an sRNA, wherein the first nucleic acid sequence and the second nucleic acid sequence are comprised in at least two fingerloop structures.

In some embodiments, the chimeric nucleic acid encodes for a chimeric small regulatory RNA. In some embodiments, the first and second nucleic acids are positioned in antisense fingerloop regions of a gene encoding a small regulatory RNA of the cell. In some embodiments, the first plasmid comprises an inducible promoter operably linked to the chimeric nucleic acid. The chimeric (fingerloop) nucleic acids disclosed herein can be used in various prokaryotic cells. In some embodiments, the chimeric (fingerloop) nucleic acids are used to test exogenous sRNAs in E. coli. In some embodiments, an sRNA of the first nucleic acid sequence binds to an mRNA of the first gene leader sequence. In some embodiments, an sRNA of the second nucleic acid sequence binds to an mRNA of the second gene leader sequence.

In some embodiments, the first reporter gene encodes a fluorescent protein. In some embodiments, the second reporter gene encodes a fluorescent protein.

In some embodiments, the chimeric nucleic acid is from about 50 to about 300 nucleotides in length. In some embodiments, the chimeric small regulatory RNA is from about 50 to about 300 nucleotides in length. In some embodiments, the first and second gene leader sequences target genes in the same metabolic pathway. In some embodiments, the first and second gene leader sequences target genes in different metabolic pathways.

In another aspect, disclosed herein is a method for modulating protein expression levels and/or mRNA expression levels from at least two target mRNAs in a cell simultaneously, the method comprising:
transforming the cell with a system for measuring the activity of a chimeric nucleic acid, the system comprising:
   a first plasmid comprising a chimeric nucleic acid, wherein the chimeric nucleic acid comprises a first nucleic acid sequence operably linked to a second nucleic acid sequence;
   a second plasmid comprising a first reporter gene operably linked to a first gene leader sequence; and
   a third plasmid comprising a second reporter gene operably linked to a second gene leader sequence;
   wherein the first nucleic acid sequence is present in a first fingerloop stem loop and the second nucleic acid sequence is present in a second fingerloop stem loop;
   wherein the first and second fingerloop stem loops inhibit the binding of the first and second nucleic acids to mismatched target sequences;
   wherein an sRNA of the first nucleic acid sequence binds to an mRNA of the first gene leader sequence and an sRNA of the second nucleic acid sequence binds to an mRNA of the second gene leader sequence; and
measuring the protein expression levels and/or mRNA expression levels of the first reporter gene and the second reporter gene.

In some embodiments, the chimeric nucleic acid encodes for a chimeric small regulatory RNA. In some embodiments, the first and second nucleic acids are positioned in antisense fingerloop regions of a gene encoding a small regulatory RNA of the cell. In some embodiments, the first reporter gene encodes a fluorescent protein. In some embodiments, the second reporter gene encodes a fluorescent protein.

In some embodiments, the chimeric nucleic acid is from about 50 to about 300 nucleotides in length. In some embodiments, the chimeric small regulatory RNA is from about 50 to about 300 nucleotides in length. In some embodiments, the cell is an Escherichia coli (E. coli) cell. In some embodiments, the cell is a Bacillus subtilis (B. subtilis) cell. In some embodiments, the cell is a Clostridium acetobutylicum (C. acetobutylicum) cell. In some embodiments, the cell can be any suitable prokaryotic cell.

In some embodiments, the chimeric nucleic acid binds to the at least two target mRNAs encoding at least two endogenous cell enzymes, and wherein binding results in a reduction of activity in the cell of the at least two cell enzymes. In some embodiments, the reduction in activity occurs due to the decrease in translation (and does not affect the enzyme's rate of activity directly). In some embodiments, the chimeric nucleic acid binds to the at least two target mRNAs encoding at least two heterologous cell enzymes. In some embodiments, the chimeric nucleic acid binds to the at least two target mRNAs encoding at least two endogenous cell enzymes.

In some embodiments, the at least two target mRNAs are in the same metabolic pathway. In some embodiments, the at least two target mRNAs affect different metabolic pathways. For example, the systems herein can be used to alter ATP levels while improving yield of a specific metabolite in a different pathway.

In some embodiments, the reporter gene encodes a GFP protein. In some embodiments, the reporter gene encodes an mCherry protein.

In another aspect, disclosed herein is a method for modulating mRNA expression levels from at least two target mRNAs in a cell simultaneously, the method comprising:
transforming the cell with a system for measuring the activity of a chimeric nucleic acid, the system comprising:
   a first plasmid comprising a chimeric nucleic acid, wherein the chimeric nucleic acid comprises a first nucleic acid sequence operably linked to a second nucleic acid sequence;
   a second plasmid comprising a first reporter gene operably linked to a first gene leader sequence; and
   a third plasmid comprising a second reporter gene operably linked to a second gene leader sequence;
   wherein the first nucleic acid sequence is present in a first fingerloop stem loop and the second nucleic acid sequence is present in a second fingerloop stem loop;
   wherein the first and second fingerloop stem loops inhibit the binding of the first and second nucleic acids to mismatched target sequences;
   wherein an sRNA of the first nucleic acid sequence binds to an mRNA of the first gene leader sequence and an sRNA of the second nucleic acid sequence binds to an mRNA of the second gene leader sequence; and
measuring the mRNA expression levels of the first reporter gene and the second reporter gene.

In some embodiments, the chimeric nucleic acid effects mRNA expression levels by modulating the stability of the target mRNA. In some embodiments, the chimeric nucleic acid effects mRNA expression levels by blocking the access of the ribosome.

In another aspect, disclosed herein is a method for modulating protein expression levels from at least two target mRNAs in a cell simultaneously, the method comprising:
transforming the cell with a system for measuring the activity of a chimeric nucleic acid, the system comprising:
   a first plasmid comprising a chimeric nucleic acid, wherein the chimeric nucleic acid comprises a first nucleic acid sequence operably linked to a second nucleic acid sequence;
   a second plasmid comprising a first reporter gene operably linked to a first gene leader sequence; and
   a third plasmid comprising a second reporter gene operably linked to a second gene leader sequence;
   wherein the first nucleic acid sequence is present in a first fingerloop stem loop and the second nucleic acid sequence is present in a second fingerloop stem loop;
   wherein the first and second fingerloop stem loops inhibit the binding of the first and second nucleic acids to mismatched target sequences;
   wherein an sRNA of the first nucleic acid sequence binds to an mRNA of the first gene leader sequence and an sRNA of the second nucleic acid sequence binds to an mRNA of the second gene leader sequence; and
measuring the protein expression levels of the first reporter gene and the second reporter gene.

### BRIEF DESCRIPTION OF THE DRAWINGS

The accompanying figures, which are incorporated in and constitute a part of this specification, illustrate several aspects described below.

FIG. 1 shows prototyping of dual-acting retargeted sRNAs. To coordinate two simultaneous interventions in a metabolic pathway, a retargeting system was developed for assaying dual-acting sRNA. (a) A particular metabolic engineering intervention can inform the choice of two target mRNAs to be tuned by coordinate regulation (e.g., improved n-butanol fermentation selectivity and yield; buk and hydA mRNAs of Clostridium acetobutylicum) using a retargeted sRNA (right). (b) Retargeted antisense sRNA “fingerloop” library variants based on the DsrA sRNA scaffold are designed to pair with these mRNA targets. The mRNAs to be tested are prepared as fusions with two fluorescent reporter genes, and effects of sRNA variants are quantified during expression in E. coli. Successful prototype sRNAs could then be introduced into the desired host organism, ideally without modification of the host genome.

FIG. 2 shows DsrA and its mode of action. (a) The secondary structure model of DsrA established by RNA footprinting. This DsrA model diagram is superposed with the location of three native DNA restriction sites in the dsrA transcript sequence (ApoI, AflII, Bsu36 I; cyan). These sites plus two sites in the vector (AatII, MfeI; gray) permit modular cloning of synthetic stem-loops as annealed oligonucleotide pairs. The rpoS' and hns' antisense regions are highlighted (gray and black). In this structure model, an extended anti-hns 5′-mRNA interaction has been included in DsrA (loop 2, white background) predicted in silico and described elsewhere. (b) DNA sequence of the dsrA gene in the sRNA-producing plasmid pSDS801a; top strand is sense (+) strand. The AatII and MfeI sites (gray) are present in the plasmid vector. (c-d) Mode of action of DsrA at rpoS and hns. (c) DsrA activates an intrinsically repressed rpoS transcript reporter fusion and (d) enhances the turnover of hns transcript fusions. Gray paired circles represent ribosomes. Circled numbers indicate RNA:RNA interactions via individual DsrA stem-loop structures 1 and 2.

FIG. 3 shows a genetic system for characterization of DsrA activity at multiple targets. All three transcripts originate from separate, compatible plasmids, and these transcripts are controlled with orthogonal repressor protein/inducer pairs. (a) Transcription of translationally cis-repressed rpoS::gfpᵤᵥ (green) is induced with anhydrotetracycline (aTet), giving low/no green fluorescence signal. Transcription of hns::mCherry (red) is induced with arabinose (Ara) and gives a strong red fluorescence signal. DsrA (above) is induced with IPTG (+) and increases translation of GFP to produce a strong green signal, whereas DsrA antagonizes the translation of mCherry (open and “flashing” filled circles). (b-c) Fluorescent protein assays validate altered reporter gene expression in the presence of DsrA. (b) DsrA enhances rpoS::gfpᵤᵥ expression and (c) antagonizes hns::mCherry expression. Control=empty vector (pSDS801a ΔdsrA hfq⁺). Asterisks indicate statistical significance, assessed by one-tailed matched pairs t-test (α=0.01).

FIG. 4 shows the tuning of gene expression using DsrA. A three-plasmid system was used to demonstrate tuning of gene expression by DsrA at both rpoS and has reporter genes. (a-b) Translation of maximally induced RpoS-GFPuv (20 ng/mL aTet) and H-NS-mCherry (2% Ara) was quantified by fluorescence at different levels of DsrA induction ([IPTG]=0-1 mM). (c-d) Surface plots demonstrate tuning of reporter gene expression using different levels of DsrA induction (0-1 mM IPTG) at different levels of rpoS::gfpᵤᵥ (0-20 ng/mL aTet) and hns::mCherry mRNA induction (0-2% Ara).

FIG. 5 shows a scheme for retargeting DsrA via fingerloop antisense-motif libraries. The location of the antisense sequences of DsrA are highlighted on a cartoon RNA structure diagram (cf. FIG. 2). The native-like “fingerloop” structure of DsrA stem loops was conserved while using larger loop regions. Boxed sequences in stem-loop 1 (panel a, middle) or stem-loop 2 (panel b, middle) indicate the location of synthetic antisense sequences in the stem-loop structure (gray uppercase “N” residues). Base pairs (black lowercase “n” residues) were added to maintain a stem-loop of approximately the same stability in the same location as wild-type DsrA. In some cases, mismatches were introduced into a stem to approximate the stem-loop 1 stability of wild-type DsrA. (a) Antisense sequences targeting the TIR of gene 1 (buk, above) were cloned to replace native DsrA stem-loop 1 with a synthetic fingerloop motif. (b) Antisense sequences targeting the TIR of gene 2 (hydA, below) were similarly cloned to replace native DsrA stem-loop 2. A series of antisense sequence “tiles” were designed to pair with the TIR sequences of target mRNAs, then were used to prepare a small antisense DsrA-variant library for each target (horizontal black bars). Each library of DsrA variants contains sequences that are antisense to the new target mRNA translation initiation region (TIR), starting at the ribosome-binding site (RBS) region of the TIR-reporter fusion construct.

FIG. 6 shows the re-targeting of individual stem loops of DsrA. (a) Stem-loop 1 of DsrA was replaced with antisense sequences targeting the buk TIR (DsrA-buk′1); separately, (b) stem-loop 2 of DsrA was replaced with antisense sequences targeting the hydA TIR (DsrA-hydA′2). (c) Reporter gene assays for DsrA-buk′1 SL1 library variants with the buk::mCherry reporter gene and (d) assays of the DsrA-hydA′2 SL2 library variants with the hydA::mCherry reporter gene. Assays are the result of 3-4 replicates, where error bars represent the standard error of the mean. Asterisks indicate statistical significance, assessed by one-tailed matched pairs t-test (α=0.01). The control experiment in 6D (vector with hfq but no sRNA gene) has a statistically significant change of low amplitude upon IPTG induction.

FIG. 7 shows the re-targeting both stem loops of DsrA simultaneously. Cartoon structure diagrams depict functional DsrA variants substituted at either (a) stem loop 1 or (b) stem-loop 2 for retargeting as described in FIG. 5. (c) Scheme for combining DsrA-buk′1 and DsrA-hydA′2 variants to make dually re-targeted DsrA-buk′1-hydA′2 variants. Stem loops 1 and 2 target the buk TIR and the hydA TIR, respectively. (d-e) Reporter gene assays for DsrA-buk′1-hydA′2 variants using reporter genes (d) buk::mCherry and (e) hydA::gfpᵤᵥ. Assays for GFPuv and mCherry activity were read simultaneously during cell growth. Assays are the result of 7 replicates, where error bars represent the standard error of the mean. Asterisks indicate statistical significance, assessed by one-tailed matched pairs t-test (α=0.01).

FIG. 8 shows the plasmid maps for the sRNA plasmid and the two reporter plasmids. (A) The sRNA plasmids are derived from pBR-plac-DsrA and contain a ColE1 origin of replication, lad repressor protein, hfq gene and ampicillin resistance gene (bla). (B) The GFPuvₘᵤₜ₆ reporter plasmids are derived from pACYC184 (NEB), and contain a p15A origin of replication, tetR repressor protein, and chloramphenicol resistance (cat) gene. (C) The mCherry reporter plasmids are derived from pBAD42 and contain a pSC101 origin of replication, lacY-A177C relaxed-sugar permease mutant, araC repressor protein and spectinomycin (spc^{R}) drug resistance gene. Variants of the pBAD reporter used in these assays also contain a p15A origin that normalizes the copy number between the two reporter genes, but the extra origin is dispensable for the assay. Vertical maps adjacent to each plasmid represent the cloning sites in the modular variants of each plasmid, used to facilitate exchange of sRNA and reporter gene sequences. Plasmid maps are not to comparable scales.

FIG. 9 shows the validation of the sRNA plasmid (pSDS801) using a colorimetric assay. Overnight cultures were diluted 1:1000 into LB media with or without IPTG. Cells were grown to OD₆₀₀˜1.0 in LB and harvested for a standard beta-galactosidase assay (Miller Assay)(Miller 1972) using the strain M182 dsrA::cat proU::lacZ. (Lease et al. 1998) The activation of proU gene transcription and subsequent translation of ProU-LacZ is due to DsrA blockage of H-NS translation; H-NS blocks proU transcription, (Gowrishankar 1985) thus proU::lacZ is an indirect readout of DsrA activity. Error bars represent standard error for three measurements taken on separate days.

FIG. 10 shows the demonstration of the robustness of the three-plasmid system and the spectral resolution of the GFPuvₘᵤₜ₆ and mCherry reporter proteins. A clear partitioning was observed of red and green fluorescent signals without fluorescent spectral overlap, independent of the level of DsrA production, using maximally-induced GFPuvₘᵤₜ₆ (20 ng/ml aTet induction) and mCherry (2% Ara induction) fusion proteins. DsrA was induced across a range of IPTG concentrations (0-1 mM). (A) RpoS-GFPuvₘᵤₜ₆ in the green channel, showing DsrA regulation of translation, and (B) minimal fluorescent signal cross-talk of GFPuv fluorescence in the red channel. (C) Minimal fluorescent signal cross-talk in the green channel from H-NS-mCherry fluorescence, and (D) H-NS-mCherry fluorescence in the red channel, reflecting DsrA translational regulation. See methods section for plate reader optical specifications.

FIG. 11 shows that retargeted DsrA variants can retain activity when the retargeting sequence is moved to a different stem-loop in DsrA. In each case the antisense sequence from one stem was re-positioned to mimic the wild-type DsrA antisense motif on the alternate stem (compare antisense sequence location in FIG. 1A, stems 1 and 2). (A) DsrA variants with anti-buk sequences in stem 1 (buk′1) were moved to the stem 2 motif (buk′2) and (B) tested on the buk::mCherry reporter gene fusion. (C) Anti-hydA sequences in stem 2 (hydA′2) were moved to the stem 1 motif (hydA′1) and (D) tested on the hydA::mCherry reporter gene fusion. (E) Stem-loop 2 fingerloops were converted to (F) stem-loop 1 fingerloops. Engineered mismatches that decrease stem stability can improve regulatory activity. Assays are the result of 4 replicates, where error bars represent the standard error of the mean. Asterisks indicate statistical significance, assessed by one-tailed matched pairs t-test (α=0.01).

FIG. 12 shows a model scheme for dual-retargeting DsrA variants. These DsrA variant secondary structures illustrate the conservation of native-like DsrA structure in DsrA variants, but the RNA structures have not been experimentally verified. (A) The wild type DsrA structure shown in FIG. 1A. (B) DsrA SL1 is retargeted as buk′1.1 by replacing the rpoS' stem loop with a synthetic anti-buk stem-loop. (C) DsrA SL2 is retargeted as hydA′2.4.1 by replacing the hns' stem loop with a synthetic anti-hydA stem-loop. (D) DsrA SL1-buk′1.1 and DsrA SL2-hydA′2.4.1 were combined to make a dually retargeted DsrA buk′1.1-hydA′2.4.1 variant. The Hfq-binding DsrA native sequence (single-stranded AU-rich region between stems 1 and 2)(Lease and Woodson 2004) and the native DsrA transcription terminator (stem-loop 3) were unaltered. Antisense sequences are in grayed capital letters. Sequences added to preserve stems are in black lowercase letters. Native DsrA sequences are in bold black capital letters.

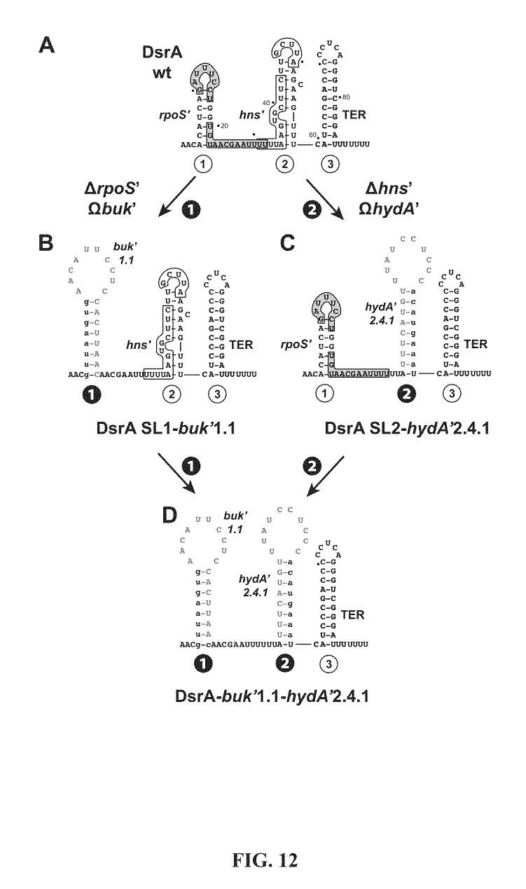

FIG. 13 shows a diagram of a DsrA sRNA derivative showing two abstract fingerloops in the context of the DsrA scaffold. Stems 1 and 2 are synthetic fingerloop sequences that adopt their characteristic structure. The third stem-loop of this DsrA sRNA derivative is the native rho-independent terminator of transcription, and can also be replaced with different terminator sequences. N=antisense, n=complement or mismatch nucleotide (compare FIG. 11F) which can be varied to alter the stem stability and thus the regulation parameters. Bulged nucleotides (not shown) can also be incorporated into the complement strand of a fingerloop to scale stem stability. Restriction sites exist at the DNA level. RNA sequences (binding sites) shown in magenta bind the Hfq protein in the pore and rim high-affinity sites.

FIG. 14 shows a schematic of the optimization of the DsrA scaffold by varying the length of the loops and stems of the fingerloop.

FIG. 15 shows covariation of the loop and stem maintain antisense sequence.

FIG. 16 shows 5 fingerloop scaffolds with a wide range of free energies of stability (above) together with their corresponding 5 near-equivalent RNA, free energy decreased scaffolds (below).

FIG. 17 shows the level of fluorescence for 5 optimized near equivalent RNA, free energy decreased scaffolds.

FIG. 18 shows that the stem length can play a role in the extinction of activity.

FIG. 19 shows a schematic of toeholds.

FIG. 20 shows examples of toehold variants and that said variations maintain the same antisense sequence.

FIG. 21 shows 6 examples of fingerloop scaffolds with varying toehold lengths and with a wide range of free energies of stability (above) together with their corresponding 6 near-equivalent RNA, free energy decreased scaffolds (below).

FIG. 22 shows the level of fluorescence for 6 optimized near equivalent RNA, free energy decreased scaffolds.

FIG. 23 shows a schematic of a hybridization filtering experiment and results for structured and unstructured equivalent RNAs.

FIG. 24 shows compensatory mutagenesis validates RNA:RNA interactions. (A) Position of mismatches tested against compensatory mutants in the reporter gene series. (B) Heat map of fold-effect regulation.

FIG. 25 shows that toeholds enable some filtering.

### DETAILED DESCRIPTION

The inventors have developed novel systems and methods to measure the activity of chimeric nucleic acids in a cell and methods of measuring and modulating protein expression levels and/or RNA stability from multiple mRNAs in a cell simultaneously. The inventors have retargeted the small regulatory RNA DsrA to act at two or more alternative mRNA transcripts by retargeting the stem-loop antisense motifs. The inventors have developed the stem-loop antisense motifs of DsrA into a novel modular, general purpose RNA antisense-encoding structure. In some embodiments, transcription of each gene (one chimeric nucleic acid and two mRNAs) is separately and orthogonally controlled with small molecule inducers.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

As used herein, the article “a,” “an,” and “the” means “at least one,” unless the context in which the article is used clearly indicates otherwise.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 150 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. In some embodiments, the polynucleotide is composed of nucleotide monomers of generally greater than 100 nucleotides in length and up to about 8,000 or more nucleotides in length.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein. The term “regulatory element” is intended to include promoters, enhancers, internal ribosomal entry sites (IRES), translation initiation regions (TIRs), and other expression control elements (e.g. transcription termination signals, such as polyadenylation signals and poly-U sequences). Such regulatory elements are described, for example, in Goeddel, Gene Expression Technology: Methods in Enzymology 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequent coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength of 3, an expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in the same reading frame. However, operably linked nucleic acids (e.g. enhancers and coding sequences) do not have to be contiguous. Linking can be accomplished by ligation at convenient restriction sites, Gibson synthesis, or CRISPR editing. In some embodiments, a promoter is operably linked with a coding sequence when it is capable of affecting (e.g. modulating relative to the absence of the promoter) the expression of a protein from that coding sequence (i.e., the coding sequence is under the transcriptional control of the promoter). In some embodiments, operably linked nucleic acids can include chimeric nucleic acids (wherein the linked nucleic acid sequences are not naturally fused or linked together).

The term “gene leader sequence” refers to the portion of a gene that encodes for an mRNA leader sequence. The term “mRNA leader sequence” refers to the portion of an mRNA sequence that is upstream from the start of the protein coding sequence portion of the mRNA. The gene leader sequence includes, for example, the translation initiation region (TIR).

As used herein, the term “fingerloop” refers to a structure formed by an intramolecular base pairing when a nucleotide sequence and a complementary sequence thereof is present in reverse direction in the same strand and a non-complementary sequence is present there between in the same strand. For example, fingerloops can refer to the stem-loop antisense motifs of DsrA that provide a modular, general purpose RNA antisense-encoding structure. The length of the fingerloop nucleotide sequence may be, for example, in the range from 10 nt to 150 nt, 10 nt to 100 nt, 10 nt to 75 nt, 10 nt to 50 nt, or 10 nt to 25 nt. As used herein, the term “sRNA” or “small regulatory RNA” refers to a short-length RNA, which is usually 300 or less nucleotides in length, is not generally translated into protein, and effectively inhibits the translation and/or stability of a specific mRNA by complementary binding.

The term “mismatched” or “mismatched target sequence” refers to an off-target sequence that is not perfectly complementary to the first nucleic acid or the second nucleic acid of the chimeric nucleic, acid (encoding the dual retargeted sRNA as described herein). The dual retargeted sRNA may have at least one mismatch, but can also have 2, 3, 4, 5, 6 or 7 or more mismatched nucleotides to the off-target sequence.

### Systems and Methods

In one aspect, disclosed herein is a system for measuring the activity of a chimeric nucleic acid in a cell, comprising:
a first plasmid comprising a chimeric nucleic acid, wherein the chimeric nucleic acid comprises a first nucleic acid sequence operably linked to a second nucleic acid sequence;
a second plasmid comprising a first reporter gene operably linked to a first gene leader sequence; and
a third plasmid comprising a second reporter gene operably linked to a second gene leader sequence;
wherein the first nucleic acid sequence is present in a first fingerloop stem loop and the second nucleic acid sequence is present in a second fingerloop stem loop; and
wherein the first and second fingerloop stem loops inhibit the binding of the first and second nucleic acids to mismatched target sequences.

In some embodiments, the chimeric nucleic acid encodes an sRNA comprising a fingerloop structure. In some embodiments, the first nucleic acid sequence and the second nucleic acid sequence encode an sRNA comprised in stem-loop antisense structures. In some embodiments, the first nucleic acid sequence and the second nucleic acid sequence encode an sRNA comprising at least two fingerloop structures. In some embodiments, the first nucleic acid sequence and the second nucleic acid sequence encode an sRNA, wherein the first nucleic acid sequence and the second nucleic acid sequence are comprised at least two fingerloop structures.

In some embodiments, the first nucleic acid sequence is present in a descending strand of the first fingerloop stem loop. In some embodiments, the first nucleic acid sequence is present in an ascending strand of the first fingerloop stem loop. In some embodiments, the second nucleic acid sequence is present in a descending strand of the second fingerloop stem loop. In some embodiments, the second nucleic acid sequence is present in an ascending strand of the second fingerloop stem loop.

In some embodiments, the chimeric nucleic acid encodes for a chimeric small regulatory RNA. In some embodiments, the first and second nucleic acids are positioned in antisense fingerloop regions of a gene encoding a small regulatory RNA of the cell. In some embodiments, the first plasmid comprises an inducible promoter operably linked to the chimeric nucleic acid. In some embodiments, the first plasmid an IPTG-inducible P_{LlacO-1} promoter. In some embodiments, the first plasmid further comprises a lacI repressor gene. In some embodiments, the first further comprises a copy of the hfq gene. However, any suitable inducible promoter can be used in any of the plasmids to provide a method of inducing gene expression. For example, the inducible promoter can be an arabinose-inducible sRNA. In some embodiments, the first plasmid comprises an arabinose inducible promoter operably linked to the chimeric nucleic acid. In some embodiments, the sRNA can also be placed under control of a constitutive (i.e. non-inducible) promoter. When scaling fermentation to industrial scale, it may be convenient to use promoters that are not inducible by small chemicals due to cost considerations.

In some embodiments, an sRNA of the first nucleic acid sequence binds to an mRNA of the first gene leader sequence. In some embodiments, an sRNA of the second nucleic acid sequence binds to an mRNA of the second gene leader sequence.

In some embodiments, the first reporter gene encodes a fluorescent protein. In some embodiments, the second reporter gene encodes a fluorescent protein. In some embodiments, the reporter gene is a non-fluorescent protein.

In some embodiments, the chimeric nucleic acid is from about 50 to about 300 nucleotides in length. In some embodiments, the chimeric small regulatory RNA is from about 50 to about 300 nucleotides in length. In some embodiments, the chimeric nucleic acid is from about 50 to about 100, from about 50 to about 150, from about 50 to about 200, from about 50 to about 250, or from about 50 to about 300, nucleotides in length.

In some embodiments, the first and second gene leader sequences target genes in the same metabolic pathway. In some embodiments, the first and second gene leader sequences target genes in different metabolic pathways. For example, the systems herein can be used to alter ATP levels while improving yield of a specific metabolite in a different pathway.

In another aspect, disclosed herein is a method for modulating protein expression levels from at least two target mRNAs in a cell simultaneously, the method comprising:
transforming the cell with a system for measuring the activity of a chimeric nucleic acid, the system comprising:
   a first plasmid comprising a chimeric nucleic acid, wherein the chimeric nucleic acid comprises a first nucleic acid sequence operably linked to a second nucleic acid sequence;
   a second plasmid comprising a first reporter gene operably linked to a first gene leader sequence; and
   a third plasmid comprising a second reporter gene operably linked to a second gene leader sequence;
   wherein the first nucleic acid sequence is present in a first fingerloop stem loop and the second nucleic acid sequence is present in a second fingerloop stem loop;
   wherein the first and second fingerloop stem loops inhibit the binding of the first and second nucleic acids to mismatched target sequences;
   wherein an sRNA of the first nucleic acid sequence binds to an mRNA of the first gene leader sequence and an sRNA of the second nucleic acid sequence binds to an mRNA of the second gene leader sequence; and
measuring the protein expression levels of the first reporter gene and the second reporter gene.

In some embodiments, the chimeric nucleic acid comprises a fingerloop structure. In some embodiments, the first nucleic acid sequence and the second nucleic acid sequence are comprised in stem-loop antisense structures. In some embodiments, the first nucleic acid sequence and the second nucleic acid sequence are comprised in at least two stem loop structures.

In some embodiments, the chimeric nucleic acid encodes for a chimeric small regulatory RNA. In some embodiments, the first and second nucleic acids are positioned in antisense fingerloop regions of a gene encoding an endogenous small regulatory RNA of the cell. In some embodiments, the first reporter gene encodes a fluorescent protein. In some embodiments, the second reporter gene encodes a fluorescent protein.

In some embodiments, the chimeric nucleic acid is from about 50 to about 300 nucleotides in length. In some embodiments, the chimeric small regulatory RNA is from about 50 to about 300 nucleotides in length. The fingerloop nucleic acids disclosed herein can be used in various cells.

In some embodiments, the cell is an Escherichia coli (E. coli) cell. In some embodiments, the cell is a Bacillus subtilis (B. subtilis) cell. In some embodiments, the cell is a Clostridium acetobutylicum (C. acetobutylicum) cell. In some embodiments, the cell can be any suitable prokaryotic cell. In some embodiments, the fingerloop nucleic acids are used to test exogenous sRNAs in E. coli. In some embodiments, the fingerloop nucleic acids are used to test synthetic sRNAs in E. coli (and in other prokaryotes).

In some embodiments, the chimeric nucleic acid binds to the at least two target mRNAs encoding at least two endogenous cell enzymes, and wherein binding results in a reduction of activity in the cell of the at least two cell enzymes. In some embodiments, the reduction in activity occurs due to the decrease in translation (and does not affect the enzyme's rate of activity directly). In some embodiments, the chimeric nucleic acid binds to the at least two target mRNAs encoding at least two heterologous cell enzymes. In some embodiments, the chimeric nucleic acid binds to the at least two target mRNAs encoding at least two endogenous cell enzymes. In some embodiments, the chimeric nucleic acid binds to the at least one target mRNA encoding a heterologous cell enzyme and at least one mRNA encoding an endogenous cell enzyme.

In some embodiments, the at least two target mRNAs are in the same metabolic pathway. In some embodiments, the at least two target mRNAs affect different metabolic pathways. For example, the systems herein can be used to alter ATP levels while improving yield of a specific metabolite in a different pathway.

In some embodiments, the reporter gene encodes a GFP protein. In some embodiments, the reporter gene encodes an mCherry protein.

In another aspect, disclosed herein is a method for modulating mRNA expression levels from at least two target mRNAs in a cell simultaneously, the method comprising:
transforming the cell with a system for measuring the activity of a chimeric nucleic acid, the system comprising:
   a first plasmid comprising a chimeric nucleic acid, wherein the chimeric nucleic acid comprises a first nucleic acid sequence operably linked to a second nucleic acid sequence;
   a second plasmid comprising a first reporter gene operably linked to a first gene leader sequence; and
   a third plasmid comprising a second reporter gene operably linked to a second gene leader sequence;
   wherein the first nucleic acid sequence is present in a first fingerloop stem loop and the second nucleic acid sequence is present in a second fingerloop stem loop;
   wherein the first and second fingerloop stem loops inhibit the binding of the first and second nucleic acids to mismatched target sequences;
   wherein an sRNA of the first nucleic acid sequence binds to an mRNA of the first gene leader sequence and an sRNA of the second nucleic acid sequence binds to an mRNA of the second gene leader sequence; and
measuring the mRNA expression levels of the first reporter gene and the second reporter gene.

In some embodiments, the chimeric nucleic acid effects mRNA expression levels by modulating the stability of the target mRNA.

In another aspect, disclosed herein is a method for modulating protein expression levels from at least two target mRNAs in a cell simultaneously, the method comprising:
transforming the cell with a system for measuring the activity of a chimeric nucleic acid, the system comprising:
   a first plasmid comprising a chimeric nucleic acid, wherein the chimeric nucleic acid comprises a first nucleic acid sequence operably linked to a second nucleic acid sequence;
   a second plasmid comprising a first reporter gene operably linked to a first gene leader sequence; and
   a third plasmid comprising a second reporter gene operably linked to a second gene leader sequence;
   wherein the first nucleic acid sequence is present in a first fingerloop stem loop and the second nucleic acid sequence is present in a second fingerloop stem loop;
   wherein the first and second fingerloop stem loops inhibit the binding of the first and second nucleic acids to mismatched target sequences;
   wherein an sRNA of the first nucleic acid sequence binds to an mRNA of the first gene leader sequence and an sRNA of the second nucleic acid sequence binds to an mRNA of the second gene leader sequence; and
measuring the protein expression levels of the first reporter gene and the second reporter gene.

### Fingerloop Modular Units

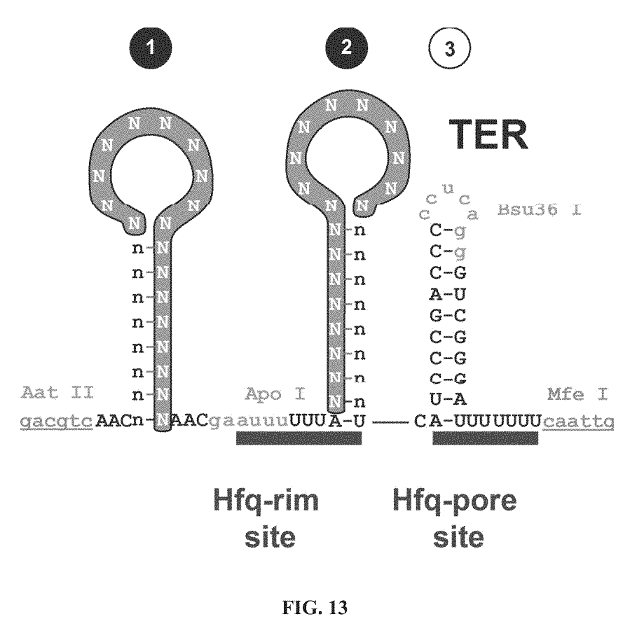

One of the important features of the fingerloop is that it acts as a modular unit of antisense sequence that can be targeted to arbitrary mRNAs or to other nucleic acids (e.g., for self-assembly of RNA/DNA nanotechnological objects or devices). As shown in FIG. 13 the fingerloop can take one of two main configurations, either with antisense sequences in the descending strand (shown in loop 1) or the ascending strand (shown in loop 2) of the helix as well as the loop region. These configurations can be reversed, swapped, or duplicated. For example, both stem-loops could use an ascending strand plus the loop sequence, leading to different combinations of fingerloops with varying efficacies against target mRNA gene expression, self-assembly, etc. The stem region is more tolerant to these mismatches in target sequences if the loop region is a perfect match. In addition, by increasing the intrinsic stem stability to make the stem structure longer, the off-target filtering efficiency can be improved. In other embodiments, fingerloop filtering is done in combination with toehold-sequence filtering. The toehold region is adjacent to the base of the stem.

### Fingerloop Parameters

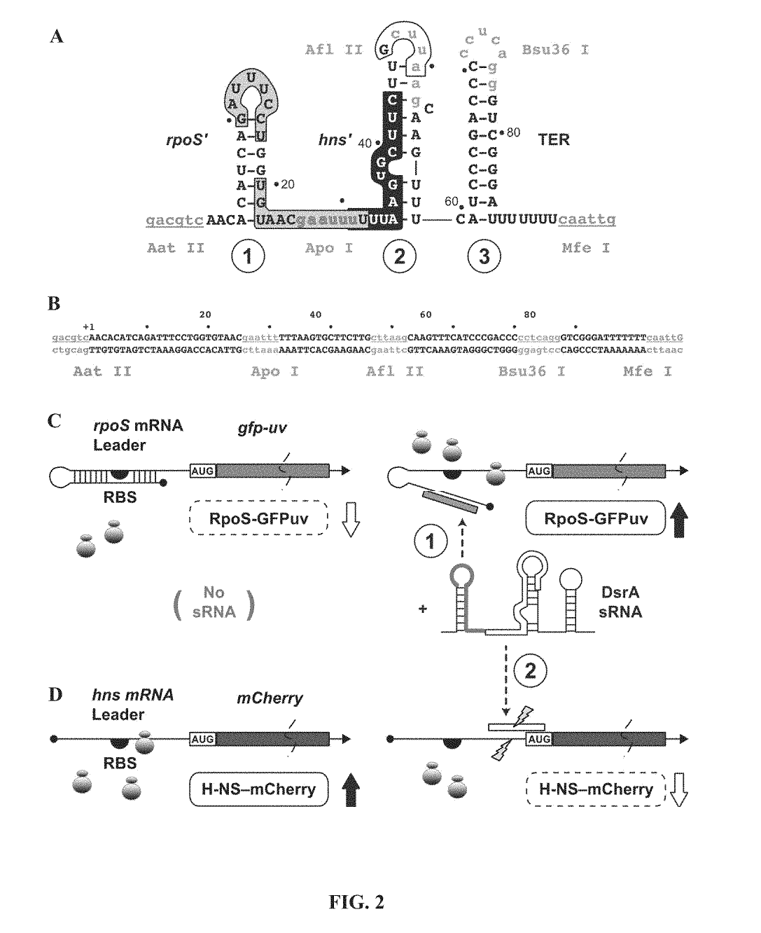

Initial experiments used an antisense “tile” size of 18 nt. In other embodiments, 24 nt of unstructured antisense sequence can be used to increase specificity. In other embodiments, tile sizes less than 18 nt can be used to diminish the dynamic range by weakening the RNA:RNA interaction. Shorter or longer antisense “tile” sequence sizes can be used in combination with the fingerloop structure to (e.g.) repress gene expression. This antisense parameter can also be used for turning on gene expression in the context of a structured, cis-repressed mRNA translation initiation region (such as the rpoS leader; FIG. 2C) that is intrinsically repressed (off-by-default) and whose expression can be activated by structural perturbation via an sRNA/fingerloop. Initial efforts have focused mostly on repressor effects of sRNAs and fingerloops because other than expressing the sRNA from a plasmid or other genetic element, no further chromosomal perturbation needs to be applied to the host cell. Any such alterations would have to be applied to the host cell before the sRNA could act as a gene expression activator. Alternatively, a gene (to be activated, or repressed) can be supplied along with the sRNA on the same plasmid or genetic element.

The length of the loop sequence in the fingerloop has been varied and it was determined that a combination of longer stem and smaller loop size eventually results in loss of gene repression by those individual fingerloop-containing sRNAs. Additional experiments can distinguish between the effects of using longer stem structures (which are more stable and may be less prone to engage in sRNA:mRNA interactions) and longer/shorter loop effects (which can act by varying the efficiency of helix nucleation in the target mRNA). The loops are also compared so that they only partially contain antisense sequence (e.g., a loop of length 10 nt that contains 6 or 7 antisense nucleotides instead of 10 antisense nucleotides) to determine efficacy, tuning of gene expression, and to determine loop structural constraints to off-target filtering.

The native DsrA sRNA contains gaps in its antisense sequence against two E. coli mRNA targets, rpoS and hns. The function of these gaps is not known, as engineered mismatches continue to function. The native DsrA contains both toehold-like and fingerloop-like antisense motifs which may tolerate rather than filter mismatches in loop antisense sequences. Thus, the effects of these mismatches are tested on sRNA kinetic and equilibrium binding of base-paired targets to examine engineered mismatches that can improve the design of fingerloops.

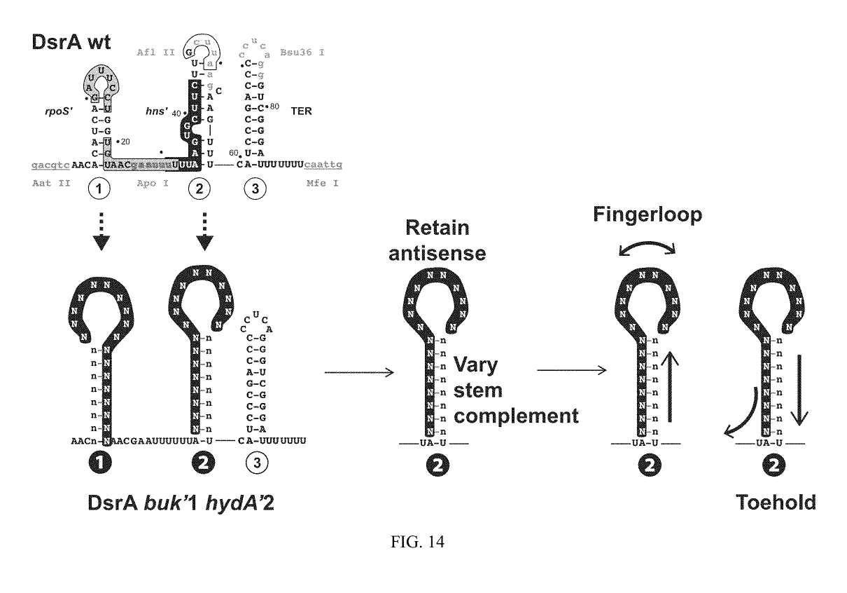

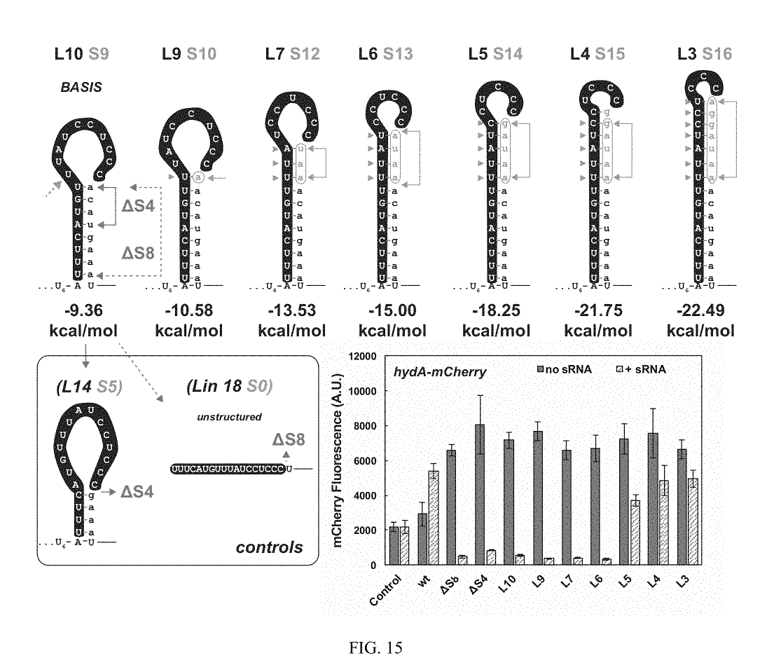

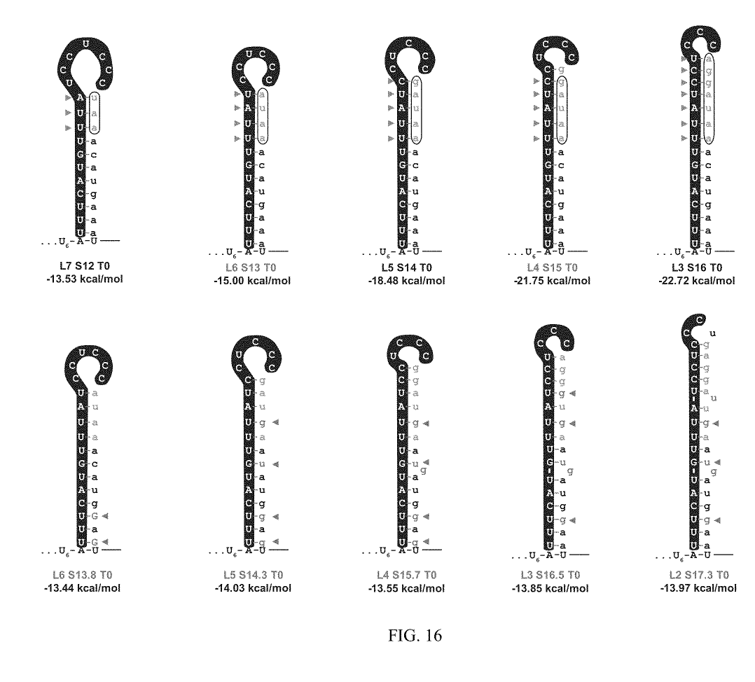

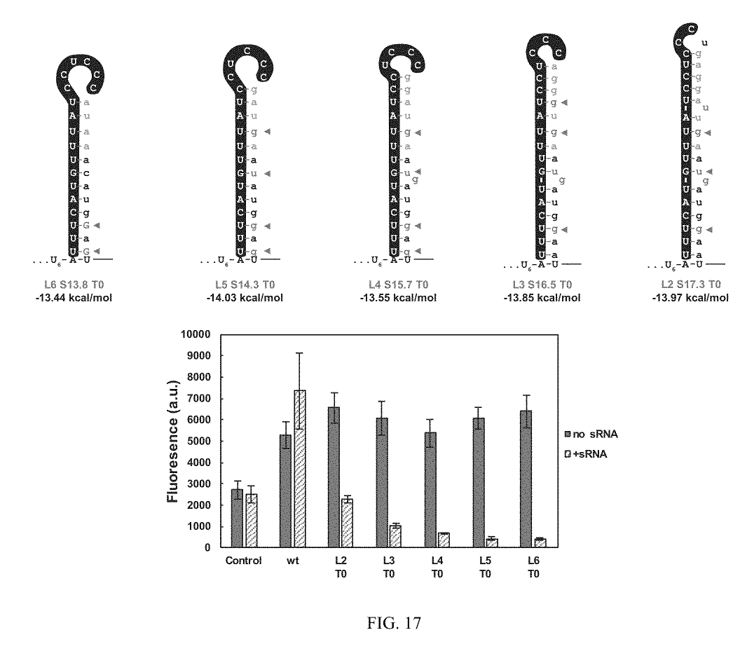

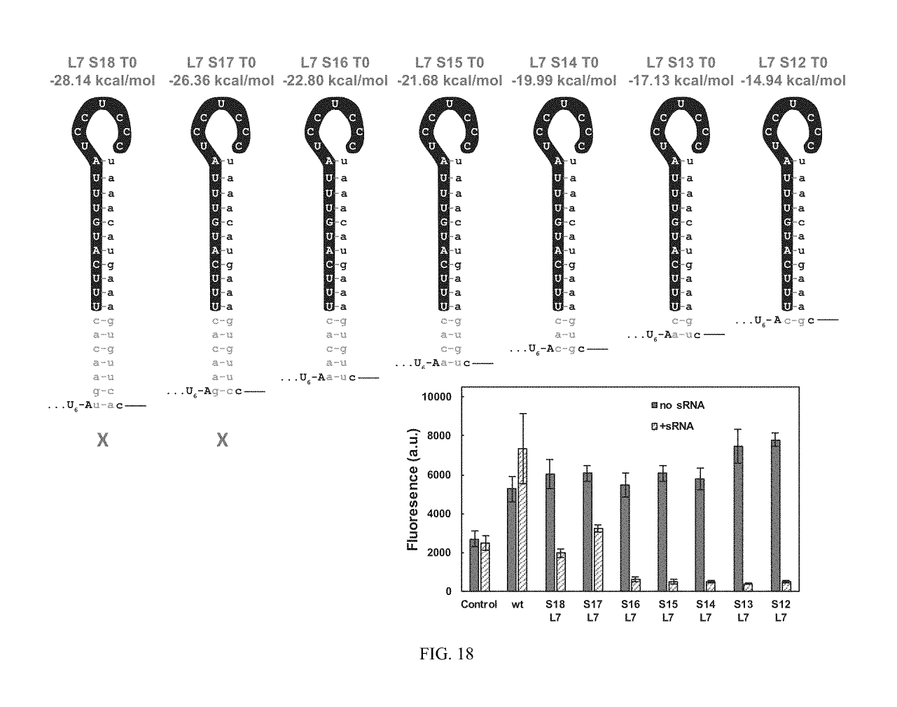

In one aspect, it is contemplated herein that the length of the loops and stems of the fingerloop can be adjusted to optimize the DsrA scaffold (FIG. 14). For example, the DsrA scaffold can be shortened, not adjusted, or extended at the loop by 1, 2, 3, 4, 5, 6, 7, 8, 9, 10, 11, 12, 13, 14, 15, 16, 17, 18, 19, or 20 nucleotides. Thus, in one aspect, the loop can be 5, 6, 7, 8, 9, 10, 11, 12, 13, 14, 15, 16, 17, 18, 19, 20, 21, 22, 23, 24, 25 nucleotides long. Similarly, the stem can be shortened, not adjusted, or extended by 1, 2, 3, 4, 5, 6, 7, 8, 9, 10, 11, 12, 13, 14, 15, 16, 17, 18, 19, or 20. The DsrA adjusting the stem and loop independently of each other (FIG. 15), but can comprise any combination of stem and loop lengths disclosed herein. The optimization would consider stem stability, stem stability as it relates to loop length or toehold length, filtering, and the ability to validate a sRNA:mRNA interaction. For example, by adjusting the stem and/or loop length of the fingerloop, fivenear-equivalent RNA, free energy decreased scaffolds were designed (FIG. 16). Five of these equivalents are shown in FIG. 17 based on fluorescence. It is further understood and herein contemplated that the length of the stem can play a role in the extinction of activity (FIG. 18). In some embodiments, the minimal functional loop size is 3 nt when stem contributions are isolated as a variable. In some embodiments, loop efficacy scales with loop size up to 5-6 nt. In some embodiments, there may be a limit of ˜−20 kcal/mole for a functional 18-mer antisense-sequence fingerloop.

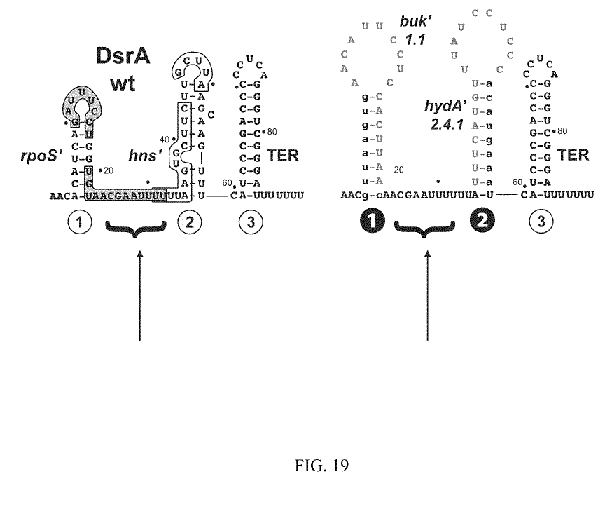

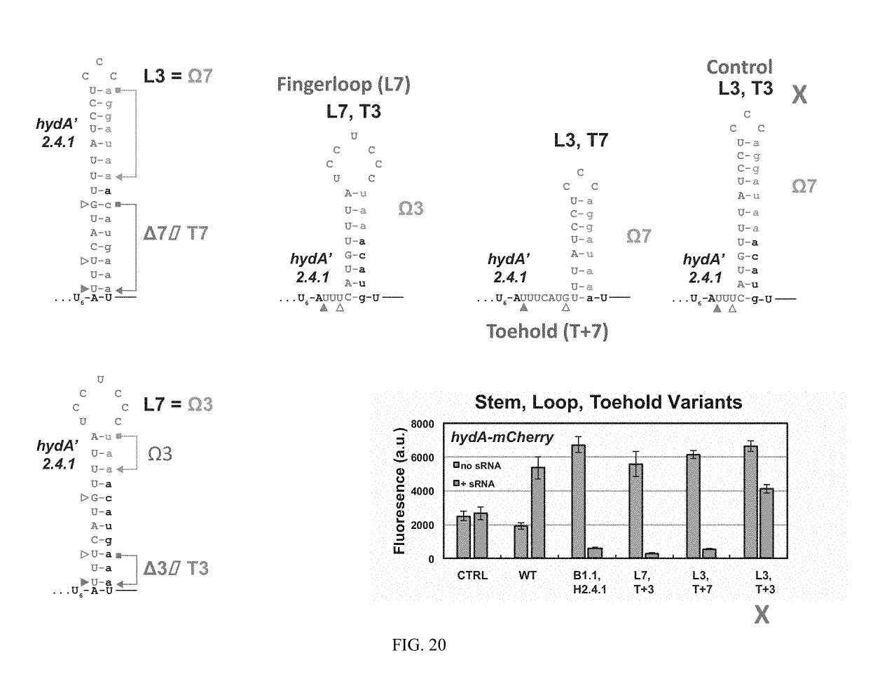

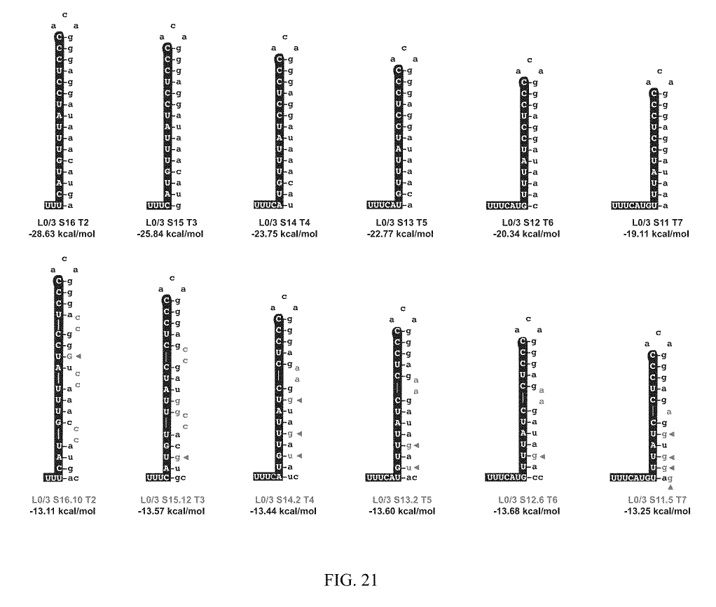

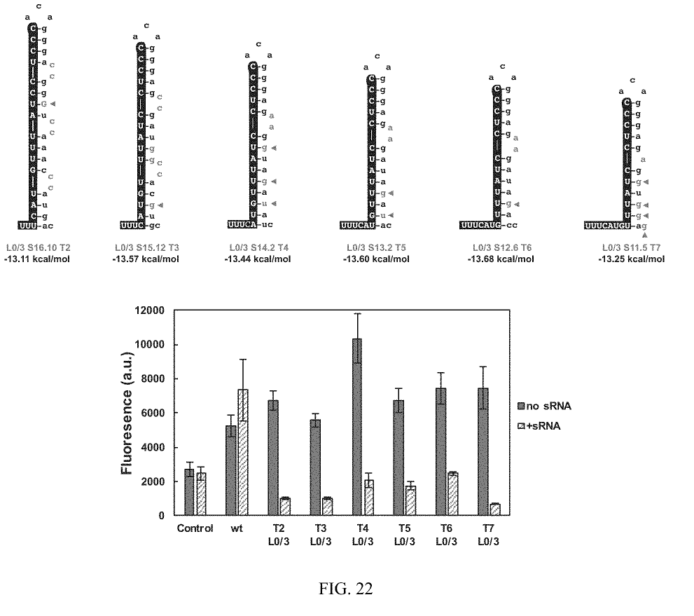

Additionally, the toehold can be varied by 1, 2, 3, 4, 5, 6, 7, 8, 9, 10, 11, 12, 13, 14, 15, 16, 17, 18, 19, or 20 nucleotides to increase stability (FIGS. 19 and 20). The toehold can be shortened, left unadjusted, or extended, independently of either or both the stem or loop of the fingerloop. FIG. 21 shows six examples of near-equivalent RNA, free energy decreased scaffolds with varying stem, loop, and/or toehold lengths. FIG. 22 shows the level of fluorescence for 6 optimized scaffolds with and without sRNA.

### Multiplexing of Fingerloops

The success of dual-acting sRNAs containing two fingerloops, and the capacity of many sRNAs to target more than two mRNAs, shows that synthetic multi-fingerloop sRNAs can be built for targeting and coordinating expression from larger numbers of genes. In some embodiments, 4-fingerloop sRNAs using the genetic system disclosed herein with fluorescent reporters are used by building the buk′ and hydA′ fingerloops onto the 5′-end of native DsrA to create an sRNA with 4-mRNA targeting (rpoS, hns, buk, hydA). These are tested in pairs using the fluorescent reporter system herein which can test two mRNA targets at a time. As an example of a different kind of natural regulatory sRNA with multiple stem-loops, the CsrB sRNA contains multiple protein-binding (rather than antisense sequence-containing) stem-loops that titrate regulatory proteins. Thus, sRNA transcripts with multiple stem-loops are tolerated by, and useful in, E. coli and other organisms, and implementing fingerloops in this context can target a number of genes simultaneously.

Applications of these single- and multi-acting sRNAs include tuning pathway gene expression for metabolic engineering of strains, for example coordinating multiple mRNAs in single or multiple/different pathways, for increasing fermentation product yields, fermentation selectivity, minimizing toxicity, and/or balancing cellular health and growth rates. Further, combinatorial knocking-down of multiple mRNAs (including essential genes that cannot be conventionally knocked out) can be used, for example, in screening drug targets in pathogens, for determining metabolic flux parameters in bacterial strains for metabolic engineering, and for producing probiotic or commensal bacterial strains.

### Hfq Protein Dependency

The basis of the Hfq interaction with the synthetic sRNAs containing fingerloops can be removed. A region at the base of the stem of wild-type DsrA also interacts with the same target genes specified in the fingerloop. This single-stranded region between DsrA stem-loop 1 and stem-loop 2 has been demonstrated to bind the Hfq protein (see FIG. 13). There are several considerations to be taken regarding this sequence/structure, and it is useful to have both Hfq-requiring and Hfq-independent synthetic sRNAs containing fingerloops.

Many, but not all sRNAs, require the Hfq protein, an RNA chaperone protein that enhances RNA:RNA interactions. The single-stranded region between DsrA stems is a binding site of Hfq. This protein probably binds most sRNAs at an internal site (such as that in DsrA) as well as at the terminator stem-loop, which ends in a poly-U tail. (see FIG. 13). Extension of sRNAs with non-native terminators having a differing composition of poly-U tails was shown to block Hfq binding of sRNAs. Moreover, enhancement of terminator stability perturbs the number of U residues following the terminator, which in turn impacts Hfq-binding and Hfq-dependent activity. Potential mechanisms for the RNA chaperone activity and mechanism of Hfq have been proposed, but in general Hfq seems to bind an sRNA and an mRNA, increasing their local effective concentration, and catalyzing their base pairing, thus culminating in the increased rate of release of an sRNA:mRNA complex.

Many sRNAs in various organisms do not require Hfq. Hfq is not present in some organisms that nonetheless use sRNA-based regulation, and so the sRNAs may rely on a different protein, or may not require a protein partner. Deletion of an internal Hfq-binding site in the E. coli sRNA RyhB did not compromise its function at a reporter gene. The Hfq site is similar to the recognition site for RNase E, a major housekeeping RNase that enhances RNA turnover in the cell. Since many sRNAs have a shorter half-life in an hfq deletion strain, it has been proposed that Hfq binds the RNase E site and sequesters the site, thus stabilizing these sRNAs. Deletion of the internal Hfq site (see FIG. 13) may stabilize sRNAs against degradation and increase their half-life.

In some embodiments, sRNAs can be used in organisms without concern for Hfq binding, or the Hfq/RNase binding site can be removed from the DsrA scaffold without compromising regulatory activity. In one embodiment, the native terminator of DsrA was replaced with a series of heterologous terminators. While most terminator variants were compromised in their regulatory activity, it is unclear if these terminators bind Hfq. Changes can also be made at the internal Hfq-binding site in sRNAs that contain either native or substituted terminators, to determine whether activity is recovered in DsrA-terminator variants and mutants that have lost activity. In this case, Hfq can stabilize DsrA derivatives but may not be required for sRNA activity at all mRNAs. DsrA binds some mRNA target derivatives in vitro with high affinity in an Hfq-independent fashion. The merit of using this type of DsrA derivative is that no provision needs to be made for producing E. coli Hfq in the desired target organism, nor need any provision be made for increasing native host Hfq protein levels, thus increasing the number of species in which the sRNA tool can be produced for customized gene regulatory activity. Overexpression of sRNAs can result in a bottleneck at Hfq which can impact growth and cause indirect sRNA-dependent effects. Overexpression of Hfq can slow cell growth rates.

Related to Hfq-independence, sequences within the DsrA scaffold are replaced to eliminate all remaining native DsrA sequences while preserving the native-like structure. Many of the DsrA derivatives produced while retargeting DsrA with fingerloops can cause a somewhat slower growth rate in E. coli. By replacing sequences in the Hfq-rim binding site, the terminator and a trinucleotide sequence near the Hfq-rim binding site, a fully-orthogonal DsrA derivative in E. coli can be used for in the methods disclosed herein.

### Toehold Sequences

The region of DsrA between stem-loops 1 and 2 forms base pairs with several native mRNA targets of DsrA. It is possible that, like the loop portion of the fingerloop, these single-stranded “toehold” regions could act as a filter of off-target sRNA:mRNA interactions in the event of a target mismatch.

This concept of a “toehold” dates from earlier work with nanoscale DNA “devices” that use DNA:DNA hybridization. Here the “toehold” region is a single-stranded region of nucleic acid that constitutes part of an antisense sequence that is complementary to a target DNA (or RNA). The other part of this antisense sequence is capable of base pairing to a target but is involved in pairing to a competitor “sink” nucleic acid strand sequence that is to be displaced. The scheme is such that “toehold” pairing to a target initiates a strand displacement reaction of the competitor in a way that would otherwise be kinetically unfavorable without the toehold. This scheme works because the nucleation of nucleic acid base pairing interactions is kinetically limiting, but the subsequent strand displacement or “branch migration reaction” is very fast. This “strand displacement reaction” then frees the full antisense sequence to pair with the target sequence at a higher final stability (lower free energy), thus the full pairing reaction is thermodynamically favorable.

In some embodiments, “toehold switches” have been used in mRNAs genetically in cis, to sense trans-acting antisense RNA signals and activate translation of target genes fused to the toehold switch, or to permit the expression of miRNA regulators. The context of toehold-mediated strand displacement can be calibrated by introducing gaps in the sequence complementarity and by adjusting other parameters. The “toehold switch” cannot act in trans, and is distinct from the idea of a toehold sequence that is complementary and thus it is distinct from trans-acting sRNAs.

Native DsrA can utilize both a toehold-mediated mechanism (likely dependent on Hfq, using the unstructured single-strand RNA sequence between stems 1 and 2) as well as a mechanism that is similar to the fingerloop hybridization, i.e., constraints on helix nucleation parameters, to increase the specificity of hybridization and diminish off-target effects. Accordingly, DsrA derivatives that combine toehold sequences (near the base of the stem-loop) with fingerloops can increase target specificity and can increase the range of gene repression activity of the sRNA. This analysis of various constructs is done in concert with removal/replacement of the native Hfq-internal binding site of DsrA, since that sequence (5′-AAUUUUUUA-3′) is located where a retargeted-toehold sequence is inserted. This native sequence is also of such low complexity as to be minimally useful for designer-retargeting purposes.

### Parameters for Fingerloops

There is a relationship between the efficacy of RNA fingerloop gene-repression activity in vivo and the free energy parameters of both the fingerloop (self-pairing) and the fingerloop-target interactions. The thermodynamic driving force for pairing of fingerloop and target is related (or proportional) to the differences in stability of the self-paired structure of the fingerloop, the self-paired structure of the target mRNA, and the final stability of the paired complex. Factors that can contribute to stability of various forms are: (a) The length and strength of the fingerloop stem sequence, including its base pair composition; (b) the existence, if any, of mismatches, bulges, G:U and G:A and other non-canonical base pairs in the stem; (c) the length, sequence composition and structure, if any, of both the antisense and target regions; (d) the length and strength of the duplex formed between the fingerloop and its target; (e) the length, strength and composition of the loop region of the fingerloop; (f) the fraction of the loop sequence that contains antisense sequences, since extra sequence could be included in this loop outside the antisense sequence; (g) the length, strength and sequence composition of a toehold sequence adjacent to the stem, and the complex formed between the toehold and the target; (h) the type, location and number of mismatches or bulged nucleotides formed between the sRNA and its target; (i) the cooperativity, if any, between the toehold:target and fingerloop:target interactions.

### Delivery of Fingerloops

RNA fingerloops have been produced in vivo (in situ) in an organism by means of producing an RNA transcript containing that RNA sequence that folds into a fingerloop.

In some embodiments, the RNA is delivered as a synthetic RNA to the cell.

In some embodiments, RNA fingerloops can be delivered to a cell by leveraging existing cellular uptake means such as endocytosis, and can be carried on a nanoparticle made of RNA, DNA or other material.

In other embodiments, RNA fingerloops can be packaged in a phage or viral capsid, envelope, liposome, or other delivery matrix, and can thereby be taken up into a cell.

### EXAMPLES

The following examples are set forth below to illustrate the systems, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Retargeting a Dual-Acting sRNA for Multiple mRNA Transcript Regulation

Multi-targeting small regulatory RNAs (sRNAs) are a useful tool for metabolic engineering applications. Natural multi-targeting sRNAs govern bacterial gene expression by binding to the translation initiation regions of protein-coding mRNAs through base pairing. An Escherichia coli based genetic system was developed to synthesize and assay dual-acting retargeted-sRNA variants. The variants can be assayed for coordinate translational regulation of two alternate mRNA leaders fused to independent reporter genes. Accordingly, the well-characterized E. coli native DsrA sRNA was used initially. The merits of using DsrA include its well-characterized separation of function into two independently folded stem-loop domains, wherein alterations at one stem do not necessarily abolish activity at the other stem. Expression of the sRNA and each reporter mRNA was independently controlled by small inducer molecules, allowing precise quantification of the regulatory effects of each sRNA:mRNA interaction in vivo with a microtiter plate assay. Using this system, DsrA variants were screened in E. coli for their ability to regulate key mRNA leader sequences from the Clostridium acetobutylicum n-butanol synthesis pathway. To coordinate intervention at two points in a metabolic pathway, bifunctional sRNA prototypes were engineered by combining sequences from two singly-retargeted DsrA variants. This approach constitutes a platform for designing sRNAs to specifically target arbitrary mRNA transcript sequences, and thus provides a generalizable tool for retargeting and characterizing multi-target sRNAs for metabolic engineering.

### Background

The use of RNA in synthetic biology applications has enormous potential. RNAs are programmable, tunable, and modular molecular tools with applications in metabolic and genome engineering, as well as in environmental, therapeutic, diagnostic and biotechnological fields. (Peters et al. 2015; Vazquez-Anderson and Contreras 2013) Using in silico secondary structure prediction tools and free-energy based simulations, RNAs can be readily designed, created and tested for specific applications. (Kushwaha et al. 2016) RNA tools have been successfully developed for altering gene expression, building genetic circuitry, and for sensing small molecules and other environmental cues. For regulatory RNAs that work by “antisense” (complementary) base-pairing interactions with other RNAs, the relationship between sequence and function can be more straightforward than for engineering protein-based regulators with novel function.

The bacterial small regulatory RNAs (sRNAs) are short RNA sequences (˜50-300 nucleotides) that alter the expression of protein-coding messenger RNAs (mRNAs) by RNA:RNA base-pairing interactions. (Gottesman and Storz 2010) Although most sRNA sequences are not conserved between species, sRNAs are a phenomenon of essentially all bacteria and represent a fundamental basis of genetic regulation. (Wagner and Romby 2015) Many sRNAs govern gene expression at the level of mRNA translation control (Storz et al. 2011) and are frequently found as stress response regulators, (Gottesman and Storz 2010) but also can alter metabolic flux in vivo. (Brantl and Bruckner 2014) Compared to conventional metabolic engineering approaches such as gene knockouts, the sRNAs of bacteria present the distinct advantage of being able to “tune” gene expression, modulating mRNA translation levels with relatively fine control. (Cho et al. 2015) Studies incorporating both theoretical and experimental components suggest that sRNA dynamics are better-suited to fine-tuning gene expression when directly compared to transcription factors. (Hussein and Lim 2012) Compared to protein-based regulation of pathway flux, sRNAs can have a faster regulatory recovery time, (Hussein and Lim 2012; Kushwaha et al. 2016) and both natural (Kang et al. 2012; Li et al. 2014) and synthetic sRNAs (Cho and Lee 2016; Liu et al. 2014; Na et al. 2013; Park et al. 2013; Sakai et al. 2014; Sharma et al. 2012) have demonstrated great potential in metabolic engineering applications.

The regulatory impact of an sRNA:mRNA interaction can be tuned by modulating the concentration of the sRNA relative to that of the target mRNA, and by altering the strength of base-pairing interactions between the RNAs. (Beisel et al. 2012; Hao et al. 2011; Hussein and Lim 2012; Mitarai et al. 2009; Na et al. 2013) Degradation of the sRNA:mRNA pair is one mechanism for inhibition by sRNA, although occlusion of the ribosome-binding site by sRNA binding has also been observed to inhibit translation. (De Lay et al. 2013; Na et al. 2013) Importantly, the translational regulatory activity of sRNAs at mRNAs is stoichiometric, not catalytic. (Masse et al. 2003) This stoichiometric mechanism of regulation provides a threshold-linear dosage-response curve when targeting an mRNA with an sRNA, which is conducive to fine-tuning enzyme levels for balancing metabolic flux. The sRNA activity rises linearly with its concentration, until the mRNA population is fully sRNA-bound and saturated, with a threshold near the 1:1 molar ratio of sRNA to mRNA. (Hussein and Lim 2012; Levine et al. 2007; Mitarai et al. 2009) Taken together, these characteristics suggest that sRNA-based gene regulation may be a useful modality for “tuning” flux in metabolic engineering.

An important goal in metabolic engineering is to strike a balance between cellular requirements for viability and the market drive for ever-increasing yields of desired product molecules. This requirement is particularly true in cases where off-target carbon flux is necessary for cell survival and growth, but where that flux might be minimized during the production phase of a manufacturing process. A balance may be met through a combination of gene knockouts and the fine-tuning of metabolite production via sRNA regulation of critical enzymes in key metabolic pathways. For example, by using sRNAs in E. coli, the yield of cadaverine was improved in fermentation cultures by tuning down two essential genes (murE and ackA) that otherwise may not have been possible using conventional gene knockouts. (Na et al. 2013) A combination of gene knockouts and synthetic sRNAs has been used in B. subtilis(Liu et al. 2014) and C. acetobutylicum(Cho and Lee 2016) to block some pathways and fine-tune the expression of other pathway enzymes.

Due to their genetic activity in trans, sRNAs can be expressed from a plasmid for high-throughput optimizations of gene expression in model microbes (Liu et al. 2014; Na et al. 2013) as well as for targeting genes in industrially relevant but genetically less-tractable organisms. (Cho and Lee 2016; Tummala et al. 2003) Critical to the metabolic engineering applications of sRNAs is their capacity to be “retargeted” by altering their antisense base pairing sequences. (Ishikawa et al. 2012; Na et al. 2013) This approach may appear simple due to the frequent location of antisense sequences in unstructured, single-stranded regions of sRNAs. (Beisel et al. 2012; Park et al. 2013; Peer and Margalit 2011) In some instances, however, alterations of sRNA sequences can strongly diminish their efficacy or stability, (Ishikawa et al. 2012; Majdalani et al. 1998) whereas other perturbations are well-tolerated. (Beisel and Storz 2011; Hao et al. 2011)

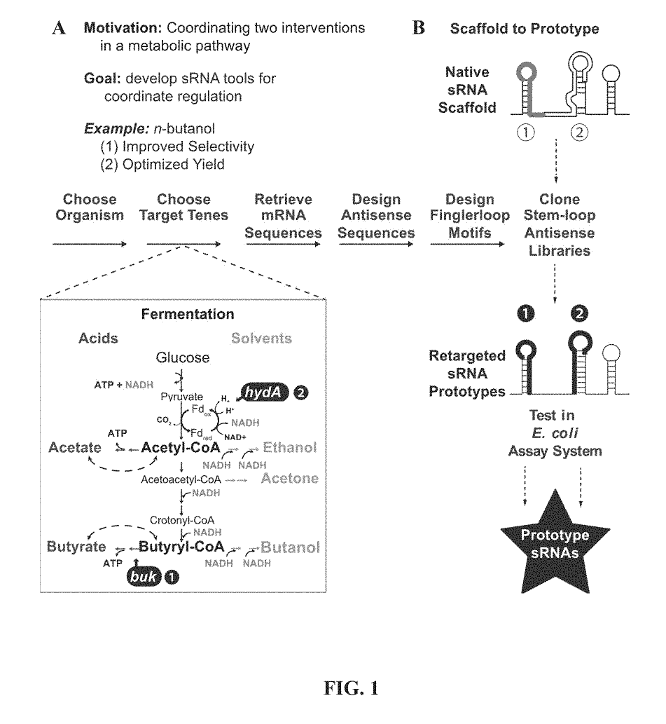

Multi-acting sRNAs could be especially useful tools for metabolic engineering because multiple mRNAs can be targeted by a single sRNA for simultaneous and coordinated translational regulation at different points in a metabolic pathway (FIG. 1). Rapid and flexible alteration of metabolic flux through multiple enzymes in an existing or engineered metabolic pathway may be optimized for yield of a desired chemical product in the context of a specific process, while maintaining cell health. (Liu et al. 2015; Nielsen 2011) Native sRNAs commonly target multiple mRNAs, (Wagner and Romby 2015) and provide sensitive thresholds for coordinated control of expression from several genes simultaneously. (Schmiedel et al. 2012) As mentioned, others have used sRNAs to target single mRNAs for metabolic engineering applications, (Cho and Lee 2016; Liu et al. 2014; Na et al. 2013) but multi-targeting sRNAs present certain advantages as tools. By using a multi-target sRNA, one sRNA agent can be introduced to create a synthetic regulatory single-input module (SIM) to coordinately regulate multiple points in a metabolic pathway. Driven from the same promoter, and using a single sRNA scaffold, the stability and level of the sRNA is similar with respect to each target. This innovation would eliminate the need to tune one sRNA promoter for each target. Rather, sRNA activity can be fine-tuned at different mRNA targets by altering the size and strength of each sRNA antisense pairing region, customized per mRNA target. (Beisel et al. 2012; Hao et al. 2011; Hussein and Lim 2012; Mitarai et al. 2009; Na et al. 2013) Systems biology simulations in silico suggest that a single-targeting or dual-targeting sRNAs can have essentially the same effect when acting at a single mRNA. (Schmiedel et al. 2012) This equivalence holds true provided that a dual-acting sRNA has similar affinities for both targets. Even when ten-fold differences exist in the sRNA:mRNA affinities for its multiple target mRNAs, the strong coordination of regulation is relatively insensitive to the sRNA:mRNA binding strength. These simulations show that multi-acting sRNAs can be used as metabolic engineering tools as the coordination of transcript regulation is relatively insensitive to the “tuning” of sRNA:mRNA pairing strength.

One of the best-characterized multi-target sRNAs, E. coli DsrA, can regulate the translation of two different global regulatory protein-coding mRNAs (rpoS and hns). Notably, DsrA base pairing to these two mRNA targets is mediated by antisense sequences in two structurally discrete stem-loop helices of DsrA (FIG. 2A-B). (de Almeida Ribeiro et al. 2012; Lalaouna et al. 2015; Lease and Belfort 2000; Lease and Woodson 2004) In the case of rpoS regulation, the DsrA stem loop 1 interacts with the cis-repressed 5′ untranslated region of the rpoS mRNA, which encodes the RpoS stationary phase/general stress response sigma factor (σ^{s}). (Majdalani et al. 1998) Base pairing by DsrA enhances RpoS expression via structural rearrangement of the rpoS mRNA leader that exposes the Shine-Dalgarno (SD) sequence for ribosome binding (FIG. 2C). (Lease and Woodson 2004) In the case of hns regulation, DsrA binds the 5′-mRNA translation initiation region (TIR) of the hns mRNA, which encodes the global transcription-silencing and nucleoid-structuring protein H-NS. (Lease et al. 1998; Majdalani et al. 1998) By contrast to DsrA stabilizing and enhancing rpoS mRNA translation, DsrA drastically decreases the hns mRNA half-life by interfering with ribosome binding and recruiting the RNase E ribonuclease, resulting in decreased H-NS translation and enhanced hns mRNA turnover (FIG. 2D). (Cayrol et al. 2015; Lalaouna et al. 2015; Lease et al. 1998) Simultaneous and coordinated DsrA activity at rpoS and hns has profound effects on the cell, altering hundreds of transcripts that affect outer membrane proteins, acid resistance mechanisms and virulence. (Lalaouna and Masse 2016; Lease et al. 2004)

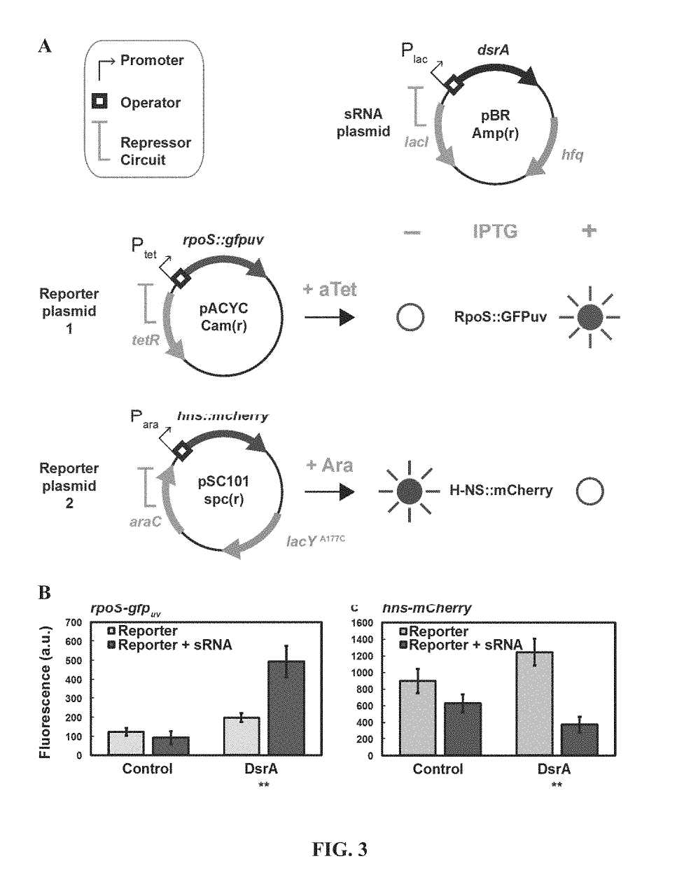

In this example, disclosed herein are methods for the retargeting of DsrA to act at two alternative mRNA transcripts, using rational design principles to retarget its two stem-loop antisense motifs. Using DsrA as a scaffold, an E. coli-based dual reporter fluorescence system was constructed for analysis of sRNAs that act at two mRNA targets. Using this three-plasmid system (FIG. 3A), transcription of each gene (one sRNA and two mRNAs) is separately and orthogonally controlled with small molecule inducers. The translation of two mRNAs with and without sRNA was assayed quantitatively via fluorescent protein expression in vivo. This genetic system was validated for sRNA-dependent tuning of translation at two native mRNA leaders, and retargeted sRNA variants were created with novel regulatory activity against two non-native mRNA leader sequences. The choice of these non-native mRNAs shows the utility in metabolic engineering, via dual-acting retargeted sRNAs, of n-butanol synthesis in the ABE fermentation pathway of Clostridium acetobutylicum.

### Results

Development of a Three Plasmid System for Screening Engineered sRNA Variants.

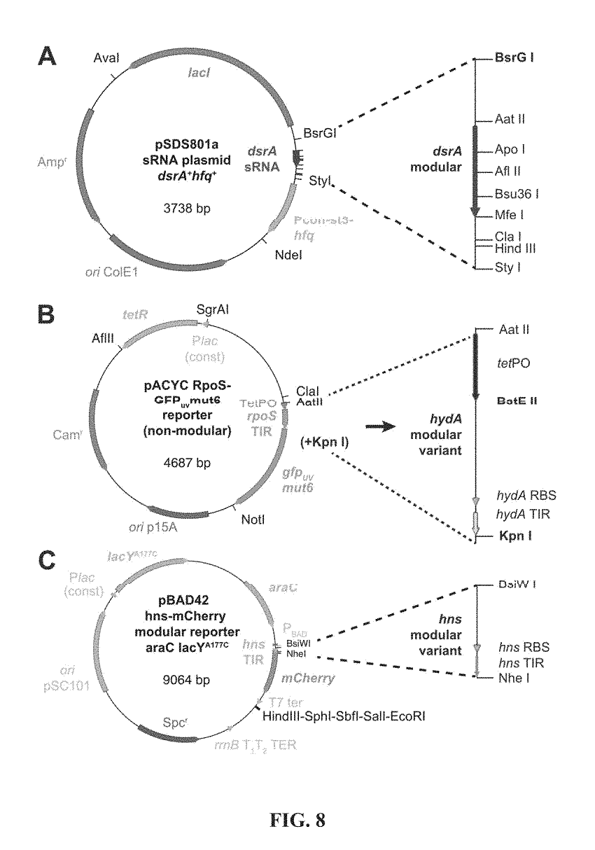

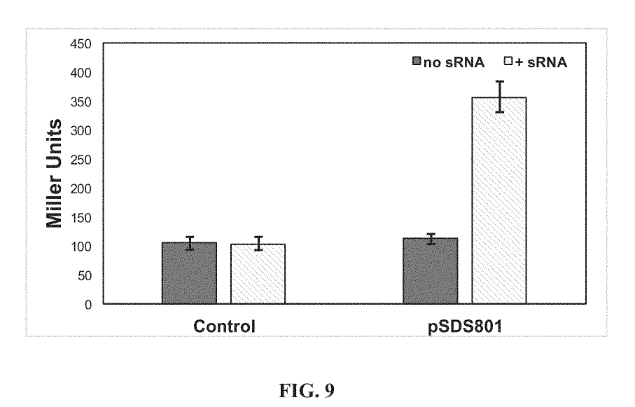

To create a modular sRNA-dual mRNA assay system for metabolic engineering, an existing DsrA-producing plasmid (pBR-plac-DsrA)(Mandin and Gottesman 2010) was modified. The previous plasmid contains dsrA transcription under the control of an IPTG-inducible P_{LlacO-1} promoter and produces the native sRNA without sequence modifications. This plasmid was altered to make it a modular cloning vector by altering one restriction site, adding a lacI repressor gene to improve sRNA transcript repression, and including a copy of the hfq gene to facilitate sRNA:mRNA interactions and mitigate an anticipated scarcity of the sRNA-binding Hfq protein in the cell (Hussein and Lim 2011) (FIG. 2A-B, FIG. 3A, and FIG. 8, panel A). This modular cloning plasmid was validated for DsrA inhibition of H-NS activity at proU-lacZ, as described previously (FIG. 9). (Lease et al. 1998)

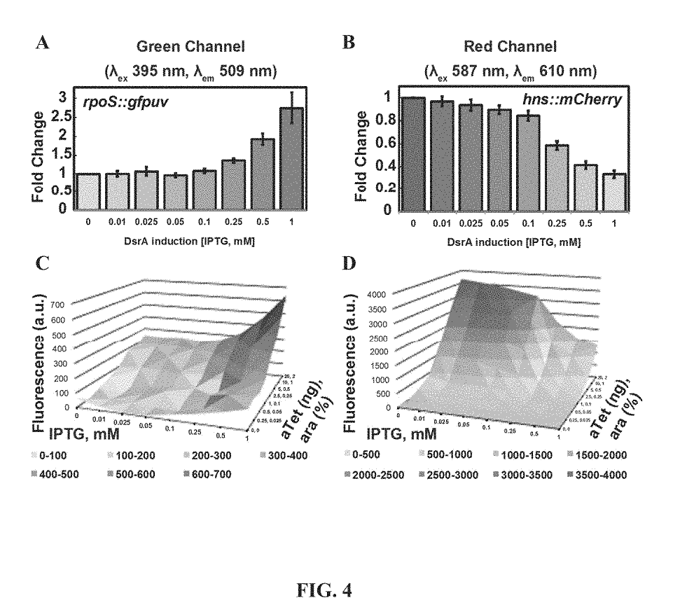

For construction of the fluorescent reporter proteins, the rpoS and hns mRNA leaders and coding sequences were fused in-frame to GFPuv and mCherry fluorescent reporter genes, respectively, under independent, inducible promoter control (FIG. 3A). Cells were grown in glucose-supplemented M9 (M9+GM) minimal medium to enable quantitation of both GFPuv and mCherry fluorescence during cell growth. The activity of DsrA at each fluorescent reporter was measured simultaneously by the change in reporter gene expression (FIG. 3B-C), and was consistent with previous individual assays of DsrA acting at rpoS and has (FIG. 2C-D). (Lease et al. 1998; Majdalani et al. 1998) When transcription of each reporter mRNA was maximally induced with anhydrotetracycline (aTet) or arabinose (Ara), simultaneous changes were observed in both RpoS-GFPuv and H-NS-mCherry protein levels, measured over a range of DsrA levels (induced with IPTG; FIG. 4A-B and FIG. 3A). Next, a plate reader was used to scale this in vivo assay to a 96-well format, and to systematically vary both reporter gene and DsrA induction levels. The resulting fluorescence surface-response plot (FIG. 4C-D) demonstrates for the first time that this system can tune expression of two target mRNAs by varying both reporter mRNA and sRNA expression. Previous examples of sRNA tuning have been obtained by altering the base pairing between sRNA and mRNA and by varying sRNA levels relative to either a single mRNA reporter level held constant, (Hussein and Lim 2012; Mitarai et al. 2009) or varied by induced transcription control. (Levine et al. 2007) This constitutes the first genetic system that is able to both quantify and tune the simultaneous activity of one sRNA at two mRNA targets.

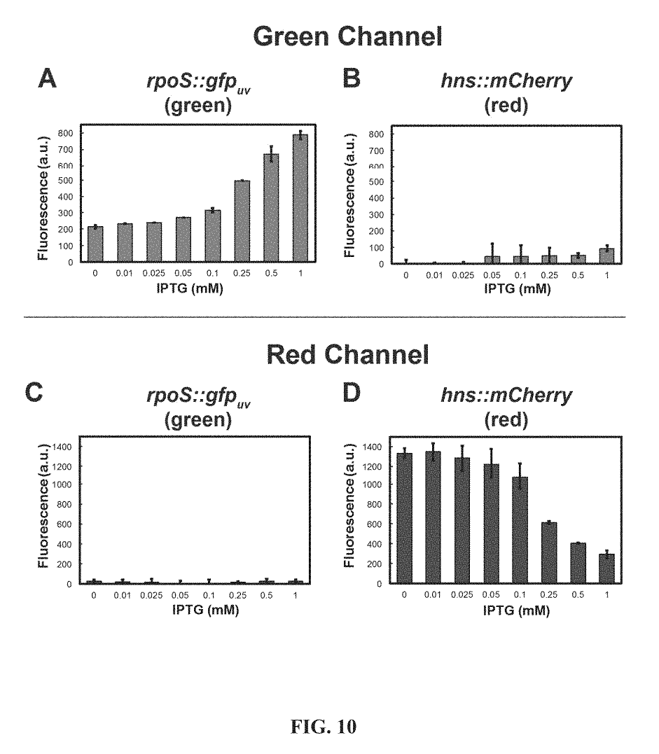

Several control experiments were performed to validate this new system. It was confirmed that the mCherry and GFPuv fluorescence could be measured independently without any significant fluorescent-signal spectral overlap (FIG. 10). Flow-cytometry was also used to verify that the gene expression behavior in bulk cultures was the result of consistent changes in fluorescence in individual cells throughout the cell population, as opposed to full induction of only partial populations of cells in the culture. There is a strong utility for this defined system, as there are many sRNAs in nature that act on multiple targets. (Wagner and Romby 2015) FIG. 4 shows the benefit of a dual acting sRNA in that arbitrary expression levels of both mRNAs can be coordinated from one sRNA.

Retargeting Stem-Antisense sRNA Variants to Non-Native mRNA Reporter Genes.

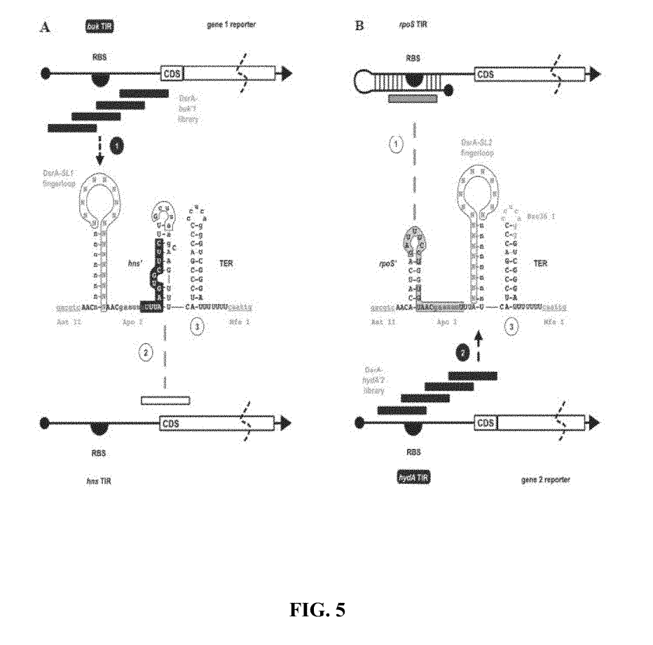

To use DsrA as a scaffold for multi-target metabolic engineering, DsrA was retargeted to bind two non-native mRNA targets in vivo in E. coli (FIG. 1). The TIR sequences of two genes were specifically targeted. The genes encode the enzyme butyryl kinase (buk), and the hydrogen-evolving hydrogenase (hydA) enzyme of Clostridium acetobutylicum ATCC 824. These enzyme genes were chosen for creating retargeted sRNA prototypes because of their relevance to optimizing yield and selectivity of n-butanol fermentation with C. acetobutylicum (FIG. 1A, metabolic pathway, inset panel). (Girbal and Soucaille 1994; Green et al. 1996; Nakayama et al. 2008; Tashiro et al. 2007; Yu et al. 2011) Target leader sequences from the C. acetobutylicum buk and hydA mRNA TIRs were fused with fluorescent reporter genes to quantify the regulatory effect of each non-native sRNA:mRNA pair (FIG. 5). The initial rpoS-gfpᵤᵥ reporter plasmid (as utilized above) lacked convenient unique restriction sites for the exchange of TIR regions, but both the buk and hydA reporter plasmids extended the rpoS and has reporter plasmid designs to facilitate mRNA leader sequence changes via forced-cloning with restriction enzymes (FIG. 8 B-C). The sRNA plasmid and non-native mRNA reporter plasmids therefore constitute a flexible modular genetic system for sRNA studies.

To alter the sRNA for retargeting to the two clostridial-leader reporter genes, the individual DsrA stem-loops were first retargeted to each non-native mRNA target (FIG. 5). Small libraries of sRNA variants (8-12 members each) were built through the incorporation of antisense sequences within each DsrA stem-loop that base pair with the TIR of each target mRNA (Table 1).

TABLE 1
List of DsrA variants^{a}

| | | | | |
|---|---|---|---|---|
| | | | Doubling | |
| | | ΔG, calc | Time, no | Doubling |
| | | (kcal/ | IPTG | Time, + |
| DsrA variant | RNA Sequence (5′-3′) | mol)^{b} | (min)^{c} | IPTG (min) |

Reporter strain: CM1000/pSC101-P_{BAD}buk::mCherry//pACYCΔtet

| | | | | |
|---|---|---|---|---|
| AdsrA^{d} | (n/a) | (n/a) | 50 ± 3.1 | 50 ± 2.9 |
| DsrA | Aacacaucagauuuccugguguaacgaauuuuuuaagu | -6.7 | 47 ± .7 | 69 ± 2.6 |
| (wild type) | gcuucuugcuuaagcaaguuucaucccgacccccucagg | | | |
| | gucgggauuuuuu (SEQ ID NO: 1) | | | |
| DsrA buk′1.1 | aacguuaagugAACAUUCCUCCACUUAACaacg | -6.6 | 132 ± 3.5 | 107 ± 3.2 |
| | aauuuuuuaagugcuucuugcuuaagcaaguuucauccc | | | |
| | gacccccucagggucgggauuuuuu (SEQ ID NO: 2) | | | |
| DsrA buk′1.2 | aacaagugagGUUAACAUUCCUCCACUUaacga | -5.1 | 139 ± 1.6 | 101 ± 3.2 |
| | auuuuuuaagugcuucuugcuuaagcaaguuucaucccg | | | |
| | acccccucagggucgggauuuuuu (SEQ ID NO: 3) | | | |
| DsrA buk′1.3 | aacuggagaaugCAUGUUAACAUUCCUCCAaac | -6.7 | 139 ± 14.7 | 96 ± 5.6 |
| | gaauuuuuuaagugcuucuugcuuaagcaaguuucaucc | | | |
| | cgacccccucagggucgggauuuuuu (SEQ ID NO: 4) | | | |
| DsrA buk′1.4 | aacagaauguuAUACAUGUUAACAUUCCUaacg | -6.8 | 126 ± 20.5 | 98 ± 8.3 |
| | aauuuuuuaagugcuucuugcuuaagcaaguuucauccc | | | |
| | gacccccucagggucgggauuuuuu (SEQ ID NO: 5) | | | |
| DsrA buk′1.5 | aacaauguuaacUCUAUACAUGUUAACAUUaac | -6.7 | 99 ± 15. | 82 ± 9.9 |
| | gaauuuuuuaagugcuucuugcuuaagcaaguuucaucc | | | |
| | cgacccccucagggucgggauuuuuu (SEQ ID NO: 6) | | | |
| DsrA buk′1.6 | aacguuaacauUAAUCUAUACAUGUUAACaacg | -5.1 | 136 ± 3.9 | 115 ± 4.3 |
| | aauuuuuuaagugcuucuugcuuaagcaaguuucauccc | | | |
| | gacccccucagggucgggauuuuuu (SEQ ID NO: 7) | | | |
| DsrA buk′1.7 | aacauauguaUAGUAAUCUAUACAUGUUaacga | -7.3 | 123 ± 17.9 | 89 ± 7.2 |
| | auuuuuuaagugcuucuugcuuaagcaaguuucaucccg | | | |
| | acccccucagggucgggauuuuuu (SEQ ID NO: 8) | | | |
| DsrA buk′1.8 | aacauguauagUAUUAGUAAUCUAUACAUaacg | -5.7 | 131 ± 6.2 | 100 ± 1.5 |
| | aauuuuuuaagugcuucuugcuuaagcaaguuucauccc | | | |
| | gacccccucagggucgggauuuuuu (SEQ ID NO: 9) | | | |
| DsrA buk′2.1 | aacacaucagauuuccugguguaacgaauuuuuuAACA | -5 | 120 ± 4.1 | 94 ± 6.9 |
| | UUCCUCCACUUAACgagaauguucaucccgacccc | | | |
| | cucagggucgggauuuuuu (SEQ ID NO: 10) | | | |
| DsrA buk′2.2 | aacacaucagauuuccugguguaacgaauuuuuuGUU | -4.7 | 109 ± 4.4 | 101 ± 9.7 |
| | AACAUUCCUCCACUUaauguuaaucaucccgaccc | | | |
| | ccucagggucgggauuuuuu (SEQ ID NO: 11) | | | |
| DsrA buk′2.3 | aacacaucagauuuccugguguaacgaauuuuuuCAU | -5.4 | (n/d) | (n/d) |
| | GUUAACAUUCCUCCAuguaacaugcaucccgaccc | | | |
| | ccucagggucgggauuuuuu (SEQ ID NO: 12) | | | |
| DsrA buk′2.4 | aacacaucagauuuccugguguaacgaauuuuuuAUAC | -5.9 | 69 ± 17.4 | 49 ± 2.7 |
| | AUGUUAACAUUCCUacauguaucaucccgaccccc | | | |
| | ucagggucgggauuuuuu (SEQ ID NO: 13) | | | |
| DsrA buk′2.5 | aacacaucagauuuccugguguaacgaauuuuuuUCU | -4.4 | 132 ± 21.4 | 127 ± 45.6 |
| | AUACAUGUUAACAUUcauguaagacaucccgaccc | | | |
| | ccucagggucgggauuuuuu (SEQ ID NO: 14) | | | |
| DsrA buk′2.6 | aacacaucagauuuccugguguaacgaauuuuuuUAAU | -4 | 121 ± 13.5 | 187 ± 8.5 |
| | CUAUACAUGUUAACguaagauuacaucccgacccc | | | |
| | cucagggucgggauuuuuu (SEQ ID NO: 15) | | | |
| DsrA buk′2.7 | aacacaucagauuuccugguguaacgaauuuuuuUAG | -3.9 | 122 ± 4.3 | 105 ± 1.9 |
| | UAAUCUAUACAUGUUagauacuacaucccgacccc | | | |
| | cucagggucgggauuuuuu (SEQ ID NO: 16) | | | |
| DsrA buk′2.8 | aacacaucagauuuccugguguaacgaauuuuuuUAU | -3.8 | 110 ± 12.6 | 97 ± 8.9 |
| | UAGUAAUCUAUACAUauuauuaauacaucccgacc | | | |
| | cccucagggucgggauuuuuu (SEQ ID NO: 17) | | | |

Reporter strain: CM1000/pSC101-P_{BAD}hydA:mCherry//pACYCΔtet

| | | | | |
|---|---|---|---|---|
| AdsrA^{d} | (n/a) | (n/a) | 54 ± 0.6 | 54 ± 0.1 |
| DsrA | (as above) | (as | 64 ± 5.5 | 65 ± 2.3 |
| (wild type) | | above) | | |
| DsrA hydA2.1 | aacacaucagauuuccugguguaacgaauuuuuuaGUU | -2.60 | (n/d) | (n/d) |
| | UAUCCUCCCAAAAUGguauaaauucaucccgaccc | | | |
| | ccucagggucgggauuuuuu (SEQ ID NO: 18) | | | |
| DsrA hydA2.2 | aacacaucagauuuccugguguaacgaauuuuuuaAUG | -2.80 | 121 ± 5.5 | 104 ± 3.4 |
| | UUUAUCCUCCCAAAAauaaauauucaucccgaccc | | | |
| | ccucagggucgggauuuuuu (SEQ ID NO: 19) | | | |
| DsrA hydA2.3 | aacacaucagauuuccugguguaacgaauuuuuuaUCA | -2.70 | 57 ± 1.2 | 70 ± 4.1 |
| | UGUUUAUCCUCCCAAaaacauuaucaucccgacc | | | |
| | cccucagggucgggauuuuuu (SEQ ID NO: 20) | | | |
| DsrA | aacacaucagauuuccugguguaacgaauuuuuuaUCA | -7.30 | 122 ± 6.4 | 101 ± 5.1 |
| hydA2.3.1 | UGUUUAUCCUCCCAAaaacaugaucaucccgacc | | | |
| | cccucagggucgggauuuuuu (SEQ ID NO: 21) | | | |
| DsrA hydA2.4 | aacacaucagauuuccugguguaacgaauuuuuuaUUU | -2.00 | 135 ± 3.3 | 124 ± 0.9 |
| | CAUGUUUAUCCUCCCacauuaaaucaucccgacc | | | |
| | cccucagggucgggauuuuuu (SEQ ID NO: 22) | | | |
| DsrA | aacacaucagauuuccugguguaacgaauuuuuuaUUU | -6.90 | 124 ± 4.1 | 109 ± 3.8 |
| hydA2.4.1 | CAUGUUUAUCCUCCCacaugaaaucaucccgacc | | | |
| | cccucagggucgggauuuuuu (SEQ ID NO: 23) | | | |
| DsrA hydA2.5 | aacacaucagauuuccugguguaacgaauuuuuuaGUU | -6.60 | 103 ± 16.5 | 102 ± 1.7 |
| | UUCAUGUUUAUCCUCaugaaaauucaucccgacc | | | |
| | cccucagggucgggauuuuuu (SEQ ID NO: 24) | | | |
| DsrA hydA2.6 | aacacaucagauuuccugguguaacgaauuuuuuaUUG | -5.30 | 125 ± 1.8 | 99 ± 2.7 |
| | UUUUCAUGUUUAUCCgaaaauaaucaucccgacc | | | |
| | cccucagggucgggauuuuuu (SEQ ID NO: 25) | | | |
| DsrA hydA2.7 | aacacaucagauuuccugguguaacgaauuuuuuaUAU | -4.70 | (n/d) | (n/d) |
| | UGUUUUCAUGUUUAUaaacaauaucaucccgacc | | | |
| | cccucagggucgggauuuuuu (SEQ ID NO: 26) | | | |
| DsrA hydA2.8 | aacacaucagauuuccugguguaacgaauuuuuuaAUU | -4.70 | (n/d) | (n/d) |
| | AUUGUUUUCAUGUUUacaauaauucaucccgacc | | | |
| | cccucagggucgggauuuuuu (SEQ ID NO: 27) | | | |
| DsrA hydA2.9 | aacacaucagauuuccugguguaacgaauuuuuuaAGA | -3.70 | 53 ± 0.3 | 53 ± 0.6 |
| | UUAUUGUUUUCAUGUaauaauuuucaucccgacc | | | |
| | cccucagggucgggauuuuuu (SEQ ID NO: 28) | | | |
| DsrA | aacacaucagauuuccugguguaacgaauuuuuuaUAA | -6.60 | 112 ± 6.1 | 121 ± 2.7 |
| hydA 2.10 | GAUUAUUGUUUUCAUuaaucuuaucaucccgacc | | | |
| | cccucagggucgggauuuuuu (SEQ ID NO: 29) | | | |
| DsrA hydA′1.3 | aacauugggaggauUCAUGUUUAUCCUCCCAAu | -14.90 | 54 ± 1.2 | 64 ± 0.6 |
| | aacgaauuuuuuaagugcuucuugcuuaagcaaguuuca | | | |
| | ucccgacccccucagggucgggauuuuuu (SEQ ID | | | |
| | NO: 30) | | | |
| DsrA | aacauugugaggauUCAUGUUUAUCCUCCCAAu | -8.30 | 118 ± 3.5 | 92 ± 2.7 |
| hydA′1.3.1 | aacgaauuuuuuaagugcuucuugcuuaagcaaguuuca | | | |
| | ucccgacccccucagggucgggauuuuuu (SEQ ID | | | |
| | NO: 31) | | | |
| DsrA | aacagggggauaaUUUCAUGUUUAUCCUCCCu | -15.30 | 140 ± 5.8 | 100 ± 1.7 |
| hydA′1.4 | aacgaauuuuuuaagugcuucuugcuuaagcaaguuuca | | | |
| | ucccgacccccucagggucgggauuuuuu (SEQ ID | | | |
| | NO: 32) | | | |
| DsrA | aacagugaggauaaUUUCAUGUUUAUCCUCCCu | -8.70 | 135 ± 7.4 | 90 ± 2.0 |
| hydA′1.4.1 | aacgaauuuuuuaagugcuucuugcuuaagcaaguuuca | | | |
| | ucccgacccccucagggucgggauuuuuu (SEQ ID | | | |
| | NO: 33) | | | |

Reporter strain: CM1000/pSC101-P_{BAD}buk::mCherry//pACYC-PₜₑₜhydA::GFPuv

| | | | | |
|---|---|---|---|---|
| AdsrA^{d} | (n/a) | (n/a) | 49 ± 2.3 | 49 ± 2.7 |
| DsrA | (as above) | (as | 99 ± 13 | 122 ± 23 |
| (wild type) | | above) | | |
| DsrA buk′1.1- | aacguuaagugAACAUUCCUCCACUUAACaacg | See | (n/d) | (n/d) |
| hydA2.3 | aauuuuuuaUCAUGUUUAUCCUCCCAAaaacau | individual | | |
| | uaucaucccgacccccucagggucgggauuuuuu (SEQ | loops | | |
| | ID NO: 34) | | | |
| DsrA buk′1.1- | aacguuaagugAACAUUCCUCCACUUAACaacg | See | 95 ± 10.4 | 81 ± 8.4 |
| hydA2.4.1 | aauuuuuuaUUUCAUGUUUAUCCUCCCacauga | individual | | |
| | aaucaucccgacccccucagggucgggauuuuuu (SEQ | loops | | |
| | ID NO: 35) | | | |
| DsrA buk′1.1- | aacguuaagugAACAUUCCUCCACUUAACaacg | See | 116 ± 9.5 | 96 ± 8.6 |
| hydA2.7 | aauuuuuuaUAUUGUUUUCAUGUUUAUaaacaa | individual | | |
| | uaucaucccgacccccucagggucgggauuuuuu (SEQ | loops | | |
| | ID NO: 36) | | | |
| DsrA buk′1.4- | aacagaauguuAUACAUGUUAACAUUCCUaacg | See | (n/d) | (n/d) |
| hydA2.3 | aauuuuuuaUCAUGUUUAUCCUCCCAAaaacau | individual | | |
| | uaucaucccgacccccucagggucgggauuuuuu (SEQ | loops | | |
| | ID NO: 37) | | | |
| DsrA buk′1.4- | aacagaauguuAUACAUGUUAACAUUCCUaacg | See | 105 ± 8.3 | 75 ± 7.9 |
| hydA2.4.1 | aauuuuuuaUUUCAUGUUUAUCCUCCCacauga | individual | | |
| | aaucaucccgacccccucagggucgggauuuuuu (SEQ | loops | | |
| | ID NO: 38) | | | |
| DsrA buk′1.4- | aacagaauguuAUACAUGUUAACAUUCCUaacg | See | (n/d) | (n/d) |
| hydA2.7 | aauuuuuuaUAUUGUUUUCAUGUUUAUaaacaa | individual | | |
| | uaucaucccgacccccucagggucgggauuuuuu (SEQ | loops | | |
| | ID NO: 39) | | | |
| DsrA buk′1.6- | aacguuaacauUAAUCUAUACAUGUUAACaacg | See | (n/d) | (n/d) |
| hydA2.3 | aauuuuuuaUCAUGUUUAUCCUCCCAAaaacau | individual | | |
| | uaucaucccgacccccucagggucgggauuuuuu (SEQ | loops | | |
| | ID NO: 40) | | | |
| DsrA buk′1.6- | aacguuaacauUAAUCUAUACAUGUUAACaacg | See | 95 ± 7.6 | 94 ± 10.3 |
| hydA2.4.1 | aauuuuuuaUUUCAUGUUUAUCCUCCCacauga | individual | | |
| | aaucaucccgacccccucagggucgggauuuuuu (SEQ | loops | | |
| | ID NO: 41) | | | |
| DsrA buk′1.6- | aacguuaacauUAAUCUAUACAUGUUAACaacg | See | (n/d) | (n/d) |
| hydA2.7 | aauuuuuuaUAUUGUUUUCAUGUUUAUaaacaa | individual | | |
| | uaucaucccgacccccucagggucgggauuuuuu (SEQ | loops | | |
| | ID NO: 42) | | | |

^{a} Native DsrA stem-loop sequences are underlined (cf. FIG. 2A). For each DsrA variant sequence, the fingerloop motif is underlined. The 18 nt antisense sequence is in underlined capitals, and the sequences that comprise the opposite side of the fingerloop stem are in underlined lower case. An additional A-U pair at the base of each fingerloop stem was retained from the wild type DsrA sequence (underlined) and was included in the NUPACK analysis.
^{b} For each fingerloop motif the minimum free energy (MFE) was calculated using NUPACK.(Zadeh et al. 2011) The MFE structure was used for DsrA wild type stem-loop 1 (rpoS′) as a reasonable basis for free energy values in synthetic fingerloops. For dual retargeted variants, see individual loop components for calculated energy values.
^{c} Doubling time was calculated from the linear regressions of the growth curves at mid-log phase where the fluorescence was read. Not determined (n/d) means the strain did not grow in M9 + GMT media.
^{d} The ΔdsrA plasmid variant of pSDS801 was used as a control, and is listed here for comparing doubling times of cultures in M9 + GMT media.

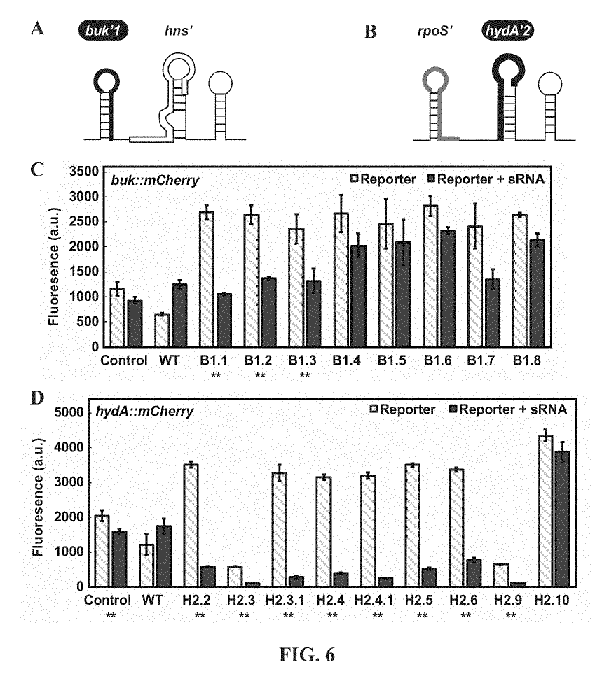

These sRNA variant libraries were designed by tiling antisense sequences (Na et al. 2013) in two or three base pair increments to pair with their putative mRNA leaders (FIG. 5, series of horizontal black bars). To create these libraries of stem-loop antisense regions, stem-loop structures 1 and 2 of DsrA (FIG. 2A) were treated as independent modules to be altered for retargeting (cf. FIG. 5A-B). The unique restriction sites in the plasmid-borne dsrA gene (FIG. 2A-B) were used for cloning pairs of annealed DNA oligonucleotides that introduce the retargeting changes in DsrA. The native dsrA anti-rpoS stem-loop 1 (SL1) sequences were replaced with sequences complementary to buk (buk′1 variants in SL1; FIGS. 5A and 6A), or native anti-hns stem-loop 2 (SL2) sequences were replaced with sequences complementary to hydA (hydA′2 variants in SL2; FIGS. 5B and 6B).

To mimic the DsrA native stem-loop antisense pattern, each 18-nt antisense sequence was placed along one side of the stem and into the loop, which was expected to create a native-like stem structure capped by an ˜8-10-nt loop region (see FIG. 5, sRNA secondary structure diagrams). For retargeted SL1 variants, the antisense sequence starts in the loop and continues down the descending portion of the stem, similar to the organization of the rpoS′ antisense sequence in wild type DsrA (FIGS. 5A, uppercase “N” nucleotides, and 6A). For SL2 variants, the antisense sequence starts at the base of the stem and continues around the top of the loop, similar to the arrangement of the hns' antisense sequence in wild type DsrA (FIGS. 5B, uppercase “N” nucleotides, and 6B). Unique sequences were then added in each variant to maintain Watson-Crick pairing in the stem helices and maintain the native-like structure of each DsrA variant stem-loop (FIG. 5A-B, lowercase “n” nucleotides). The predicted free energies of stem-loop formation of all variants were compared to verify that they did not strongly deviate from that of DsrA (analyses via NUPACK; (Zadeh et al. 2011) Table 1), and in some cases bulge mismatches were added where necessary to reduce excessive stem stability. Eight buk′1 (SL1) variants and 12 hydA′2 (SL 2) variants were designed and cloned as distinct tiled antisense sequences, sequestered in predicted synthetic stem-loop structures within DsrA (Table 1). Initially, only one stem-loop antisense sequence of DsrA was retargeted at a time while retaining the second stem-loop as a control on wild-type activity and sRNA stability. Note that the stem-loop substitutions do not alter native DsrA sequences in the single-stranded regions between stem-loops 1 and 2 (FIG. 6A-B) as this region interacts with the Hfq protein. (Lease and Woodson 2004; Panja et al. 2015) These engineered stem-loop motifs lack unstructured RNA toeholds (Green et al. 2014) and are referred to as “fingerloop” motifs.

Screening of Single-Stem Retargeted sRNA Variants.

The plasmids producing these retargeted sRNA variants were introduced into E. coli strains containing the corresponding buk and hydA fluorescent reporter genes. These strains were assayed for growth and fluorescence (GFPuv and mCherry) in a 96-well plate. Transcription of both gfpᵤᵥ and mCherry reporter gene fusions was activated by their respective small-molecule inducers, aTet and ara, with DsrA variant expression from the sRNA plasmid either induced by IPTG or left uninduced (as a control). Although the majority of these strains containing DsrA variants were able to grow in rich media (LB), most did not grow in the supplemented minimal media (M9+GM) required for simultaneous detection of GFPuv and mCherry protein fluorescence (as shown in FIG. 4). Accordingly, the minimal growth medium (M9+GM) was supplemented with 1% tryptone (M9+GMT) and the activities of the sRNA variants assessed using buk- and hydA-mCherry reporter gene fusions in the mid-log phase of growth (FIG. 6C-D). The majority of strains (17 of 20) expressing the DsrA library variants grew in this tryptone-supplemented minimal medium. A total of 3 out of 8 (37%) DsrA-buk′1 and 8 out of 12 DsrA-hydA′2 variants (67%) significantly repressed their cognate reporter protein expression (one-tailed matched pairs t-test, α=0.01). The wild-type DsrA and dsrA-deletion plasmid variants grew in all media tested with and without IPTG induction. All three of the clones that did not grow in supplemented media were DsrA-hydA′2 derivatives (hydA′2.1, 2.7, and 2.8).

Most of the DsrA derivatives have a decreased growth rate under these assay conditions when compared to wild-type DsrA or delta-dsrA controls (˜200-300% longer doubling time; Table 1). For a given retargeted DsrA variant the growth rate is comparable with and without sRNA induction (typically within ˜20-30%; see doubling times, Table 1). An analysis of variance (ANOVA) was used to determine that differences in fluorescence can be attributed to IPTG induction, and that the growth rate is not a statistically significant contributor to changes in fluorescent signal. Therefore, differences in fluorescence values with and without IPTG are likely due to sRNA activity.

The degree of change in target reporter gene fluorescence varied somewhat, depending on either the mRNA target location or the antisense sequence composition. The threshold-linear response of repression scales with the molar ratio of sRNA and mRNA, (Levine et al. 2007) and so different stabilities and thus steady-state levels of mRNA or sRNA can alter the degree of repression in each assay. DsrA-buk1 variants exhibited a low dynamic range of repression (1.8-2.5 fold decrease), whereas DsrA-hydA′2 variants repressed gene expression in a broader dynamic range (4-12.5-fold decrease). Fold-effects can reflect the copy number ratios of sRNAs to mRNAs, strengths of interaction and/or the intrinsic translation efficiency of the target mRNA. (Vazquez-Anderson and Contreras 2013) However, even modest changes in translation can have strong effects on metabolic flux, as the proteins being regulated are enzymes that exert nonlinear (catalytic) effects. A modest range of translational repression (up to ˜8-fold) is suitable for balancing metabolic flux; others have optimized repressors and trans-activating/cis-repressing sRNA:mRNA pairs for maximal fold-effects (reviewed in (Vazquez-Anderson and Contreras 2013)).

Some of the retargeted DsrA variants were deleted for native anti-rpoS and/or anti-hns antisense sequences and exhibited growth-defect phenotypes when grown in minimal medium (without tryptone). This behavior is consistent with the pleiotropic nature of DsrA activity: DsrA has 4 confirmed mRNA targets (Cayrol et al. 2015; Lalaouna et al. 2015; Lease et al. 1998) (recently reviewed (Lalaouna and Masse 2016)), with several other direct mRNA interactions predicted in silico. (Lease et al. 1998) The native DsrA promoter is strongly activated at low temperature (≤30° C.), (Sledjeski et al. 1996) and under these conditions DsrA overexpression can have manifold effects. (Lease et al. 2004) Given DsrA native function at reduced temperatures, these phenotypes were not anticipated in log-phase growth at 37° C., using cells that never reach stationary phase. These phenotypes might result from an unbalancing of coordinated DsrA effects within E. coli. In particular, RpoS and H-NS co-regulate many stress response genes, and have strong impacts on cellular physiology. (Lalaouna and Masse 2016; Lease et al. 2004)

The antisense design approach relies on the unusual antisense fingerloop motifs in DsrA. This genetic system can also be used for retargeting sRNAs to non-native mRNAs. Further, essentially any sRNA can be tested in this 3-plasmid genetic framework using single-stranded or structured antisense design, in particular using multi-acting sRNAs with the dual reporter system.

Modularity of the Stem-Antisense Motif.

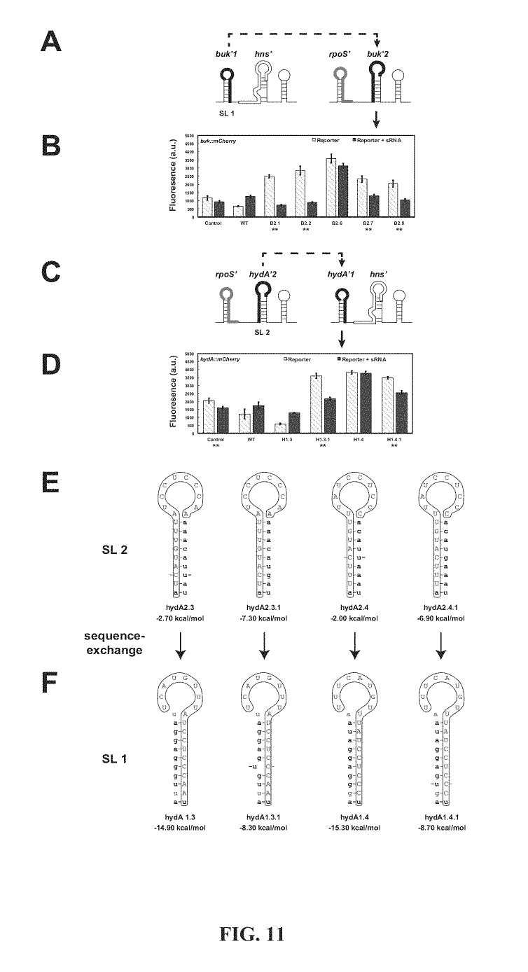

This example is the first technique to alter an sRNA antisense stem-loop control element from a positive activator (DsrA stem 1 activity at rpoS) into a negative regulator of translation. However, the activity of stem 1 as a repressor in singly-retargeted DsrA is poorer than that of retargeted stem 2. To determine whether the activity of these retargeted-antisense modules is dependent on the location within the DsrA scaffold, several retargeted antisense SL 1-buk′1 and SL 2-hydA′2 sequences were exchanged between stems 1 and 2, creating new DsrA-buk′2 (SL2) and DsrA-hydA′1 (SL1) variants. These “sequence-exchange” variants frequently retained their corresponding reporter gene repression (FIG. 11), albeit to varying degrees of activity, demonstrating apparent dependence on their sequence location within the sRNA structure. Note that exchanges of antisense regions between stems were designed to mimic the orientation of the DsrA antisense sequences, and thus were exchanged from ascending to descending (5′-3′) orientation on the stem helices during swaps (cf. FIG. 2A and FIG. 11, panels A and C).

Unexpectedly, this “sequence-exchange” experiment improved the dynamic range in some cases when moving antisense sequences from stem 1 to stem 2 (cf. FIGS. 6C and 11B, buk′1.2 moved to buk′ 2.2; buk′1.8 moved to buk′2.8). The effects of retargeting stem 1 are of lower dynamic range than those in stem 2 for the same antisense sequences in DsrA (cf. FIGS. 6C-D; cf. FIGS. 11B and 11D). These results are consistent with the finding that different subtypes of sRNAs act depending on the location of their antisense regions and the binding of both sRNA and mRNA target to the RNA “matchmaker” protein, Hfq. (Schu et al. 2015; Updegrove et al. 2016) In addition, the regulation of target mRNA seems to be dependent on the stability of the stem-loop independent of the stem-loop location within the sRNA (SL1 versus SL2). When using the identical antisense motif sequences to create slightly different structures (i.e., loop sequences move to within a stem; compare ascending and descending fingerloop motifs, FIGS. 5, 11E-F), if the stem is stabilized the sRNA regulatory strength appears to decrease, and vice versa. Comparing the predicted stability of the fingerloop motifs (Table 1) reveals that moving the antisense sequence from one stem to the other and rearranging the motif to mimic wild-type DsrA can change the stability of that stem loop (FIG. 11E-F, compare free energy values). This alteration leads to a change in regulatory effect of that antisense sequence.

Combinatorial Construction and Screening of Dual-Acting Retargeted sRNAs.

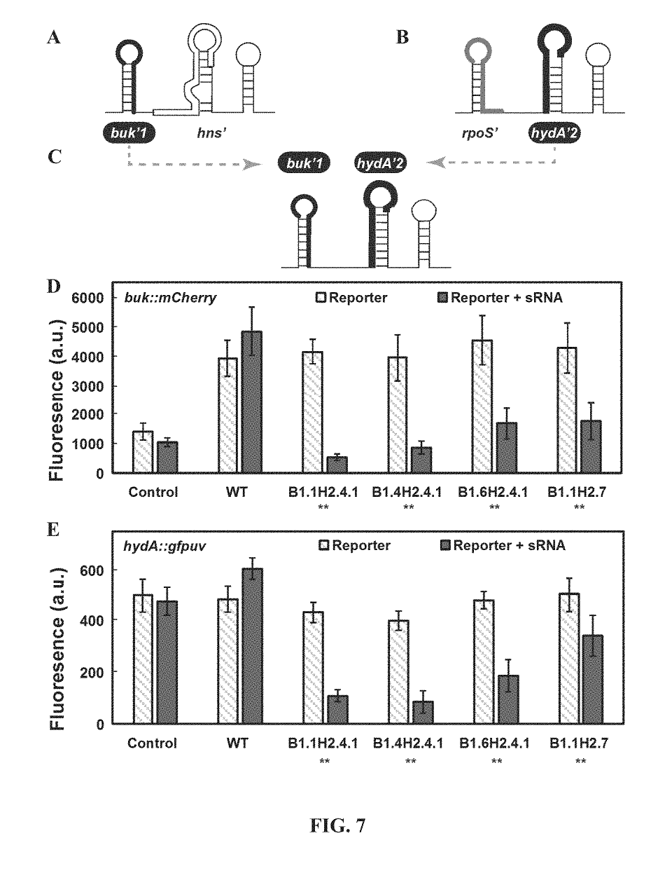

To create an engineered DsrA variant capable of coordinately targeting two different non-native mRNAs for metabolic flux control, a library was developed that combined 3 DsrA-SL1 variants (buk′1.1, 1.4 or 1.6) with 3 DsrA-SL2 variants (hydA′2.3, 2.4.1 or 2.7; FIG. 7). A hydA::gfpᵤᵥ reporter gene was also constructed in order to simultaneously quantify Buk-mCherry and HydA-GFPuv reporter fluorescence. Four out of the nine resulting dual-targeting variants (44%) significantly repressed both Buk-mCherry and HydA-GFPuv production (FIG. 7D-E). These dual-targeting variants were able to repress the buk::mCherry reporter gene expression by 2.4-7.8-fold whereas these variants exhibited a range of 1.5-4.7 fold repression of the hydA::gfpᵤᵥ reporter.

Interestingly, some retargeted stem-loops have different activities in single versus double-retargeted contexts. For example, DsrA-hydA′2.7 did not grow as a single-stem variant (SL1 rpoS′), but grew when the first stem was retargeted as buk′1.1. This result was not seen for hydA′2.7 in the context of buk′ variants 1.4 and 1.6, so the differences cannot be solely attributed to the absence of the rpoS' antisense sequence or anti-rpoS activity by SL1. DsrA-hydA′2.3 and buk′ (1.1, 1.4 and 1.6) single-stem variants were able to grow individually but when combined, none of these resulting variants were able to grow in supplemented media. It is difficult to assess the basis of these growth phenotypes based on DsrA variant stem composition alone. Levels of fluorescent signal in controls (DsrA wild type and no-sRNA controls) may be lower than expected in some experiments (FIGS. 6C-D and 7D). This variation can be explained by significantly shorter doubling times for the DsrA and delta-dsrA plasmid controls (2-3 fold faster growth; Table 1) and for two sRNA variants (hydA′2.3 and hydA′2.9, FIG. 6D).

The ability to retarget these sRNAs speaks to the robustness of sRNA structure and function. Many sRNAs target their mRNAs using unstructured regions within the sRNA. (Beisel et al. 2012; Peer and Margalit 2011) The stem-loop antisense “fingerloop” motif that was observed in DsrA stem 1 (anti-rpoS), which is also predicted for stem 2 (anti-hns)(Lalaouna et al. 2015) and validated by the retargeting data (FIGS. 6-7), includes sequences in the loop region and down only one side of the stem helix (FIG. 2A) which is a configuration not commonly seen in sRNAs. Because the opposite (cis-antisense) strand within the stem may act as a competitor for trans-antisense interactions with mRNA targets, these fingerloop motifs may contain an intrinsic hybridization-filtering function. This motif can decrease off-target mRNA binding and promote stringency and orthogonality of mRNA control due to the energetic cost of disrupting the stem-loop. Others have demonstrated strongly enhanced (10²-10³-fold) specificity of hybridization by including cis-antisense competitor or “sink” strands in the design of hybridization probes for cancer gene detection. (Wang and Zhang 2015) The ability to semi-rationally design and test synthetic sRNA regulators is validated by the high success rate for retargeting (37-67% for single-stem retargeting and ˜44% for dual targeting).

This sRNA screen has been implemented in a 96-well format to improve sample throughput relative to shaker tube growth assays. For maximal throughput using this format, ˜45 clones per day (+/−induction of sRNA) plus controls can be screened. Since the principal bottleneck is growth in the plate reader, an increase in the sample throughput can be obtained by using plate-handling robots to shuttle growing cells between a microtiter plate incubator and the plate reader. In that case, the principal bottleneck would shift to the number of plates that can be grown simultaneously. Many synthetic biology/metabolic engineering companies use robotic microtiter plate systems, and so this method is scalable to labs using such approaches. Strains could be read at a stationary phase end-point, (Urban and Vogel 2007) but this approach neglects sRNA activity in log-phase growth, which may be relevant to fermentation cultures. Antisense libraries can also be generated with tiling using larger gaps between tiles to minimize the number of sRNA variants to be screened.

Dual-Acting sRNAs as Tools.

These dual-retargeted sRNAs bind multiple targeted mRNAs, mimicking the native sRNA function for coordinating expression across operons or regulons. (Wagner and Romby 2015) Others have modified native sRNAs for metabolic engineering, (Liu et al. 2014; Na et al. 2013) but single sRNAs were required for each mRNA intervention. The methods herein can be used to implement a single multi-functional retargeted sRNA for intervention at multiple points in a metabolic pathway, using this unique system for programming multi-retargeting sRNAs based on the native DsrA antisense motifs. This goal requires transformation with an sRNA-bearing plasmid but does not require knockouts or other genome engineering. The alternative to this approach, which others have done, (Liu et al. 2014; Na et al. 2013) would be to add one single-acting sRNA per intervention, which for two interventions requires optimizing individual promoters and induction conditions for each sRNA:target pair. Implementing sRNA control in hosts with poorly-characterized genetics often means there are a limited number of characterized promoters to use in these hosts, so avoiding optimization of multiple promoters is a useful feature of the approach here. By testing dual-retargeted sRNAs in this E. coli genetic system (FIGS. 1-3), the prototyping process can be accelerated to enrich the pool of sRNAs to be tested in target organisms.

Initial experiments have focused on sRNA repressors because, unlike engineered sRNA activators, they do not require modification of the host mRNA structure. The sRNAs can also activate mRNA expression; for example, the native DsrA sRNA activates the rpoS mRNA through its interactions with a cis-repressing translational operator (FIGS. 2C and 3B). Engineering sRNA-based activation is a powerful tool that requires alteration of the mRNA leader in the chromosome. Others have modified a desired target mRNA with cis-repressor structures that, when bound by a trans-acting sRNA, convey translational activation (reviewed in (Vazquez-Anderson and Contreras 2013)). The retargeted DsrA variants herein are designed to act as negative regulators for portability to strains with minimal genetic perturbation of the host. An additional novel application of these multi-acting sRNA repressors is their use in methods to rapidly screen potential targets in a metabolic pathway in a combinatorial manner, perhaps in concert with metabolomics or other flux analyses.

A significant challenge in metabolic engineering involves moving genes and pathways from a donor organism to a more tractable host organism while retaining the control of pathway gene expression. One challenge is to balance or “tune” the carbon flux through metabolic reactions that detract from the yield or selectivity of desired product so as to improve (not necessarily maximize) production of desired chemicals. (Keasling 2008) This is particularly true in cases where a metabolic intermediate is toxic, and can be difficult if one or both organisms have genetics that are not well understood. As an example, several species of the genus Clostridium naturally produce n-butanol, a biofuel that is a useful replacement for gasoline. Transplantation of the n-butanol synthesis pathway into E. coli (Atsumi et al. 2008), yeasts and other organisms has been demonstrated, although their butanol tolerance is not high. (Knoshaug and Zhang 2009) There have been advances in genome modification via the use of mobile group II intron (targetron/clostron) technology to knock out genes in clostridia (Jang et al. 2012), but clostridial genetics are not currently well-developed for fine-tuning metabolic flux. In C. acetobutylicum others have recently used CRISPR-Cas systems for editing and gene repression (Bruder et al. 2016; Li et al. 2016) and have used synthetic antisense RNAs as metabolic flux regulators. (Cho and Lee 2016; Tummala et al. 2003)

Use of this dual-reporter system for evaluating candidate mRNA target sequences (FIGS. 1 and 3) leverages the capabilities of E. coli genetics in screening useful sRNA variants for activity regardless of the ultimate target destination species or metabolic pathway/product. The capacity of DsrA fingerloops to be semi-rationally and modularly designed with a high degree of success in retargeting is a powerful aspect of the DsrA scaffold for generating useful sRNA tools using this genetic screen. In one example, one could co-express E. coli Hfq, which was provided in this work by the sRNA vector. Additionally, it is noteworthy that C. acetobutylicum possesses both stress-response sRNAs and an Hfq protein ortholog. (Chen et al. 2011; Venkataramanan et al. 2013) Hfq is necessary for sRNA function in the related Clostridium difficile, (Bouloc and Repoila 2016) and C. difficile Hfq can complement sRNA functions in an E. coli Ahfq deletion mutant. (Caillet et al. 2014) Adding E. coli Hfq improves single-target synthetic sRNA activity in C. acetobutylicum but is not required (Cho and Lee 2016). Taken together, these findings disclose how to design and test dual-acting sRNAs in E. coli for implementation in C. acetobutylicum and/or other organisms and retain or improve sRNA function.

Hybridization Filtering Experiment

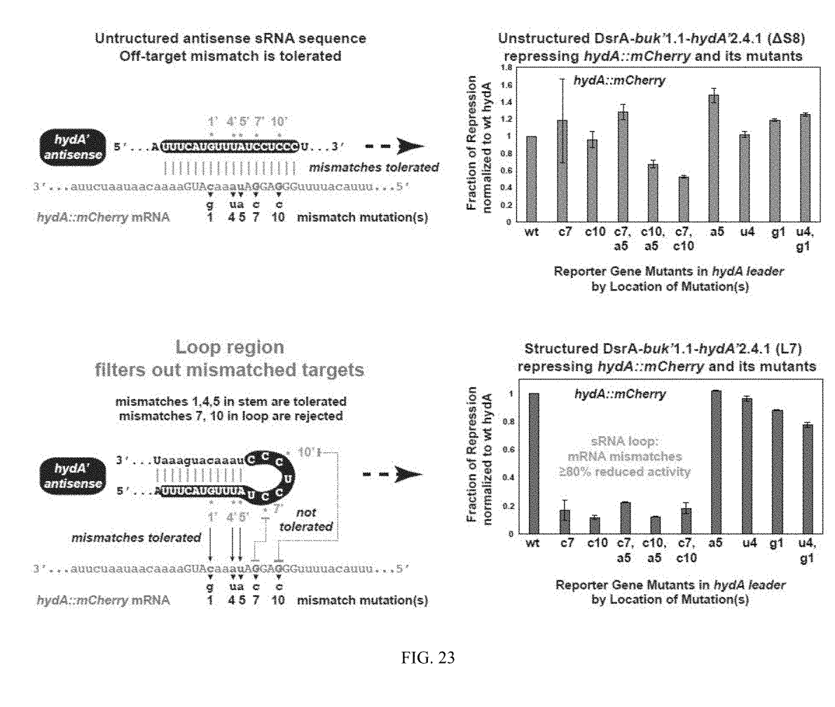

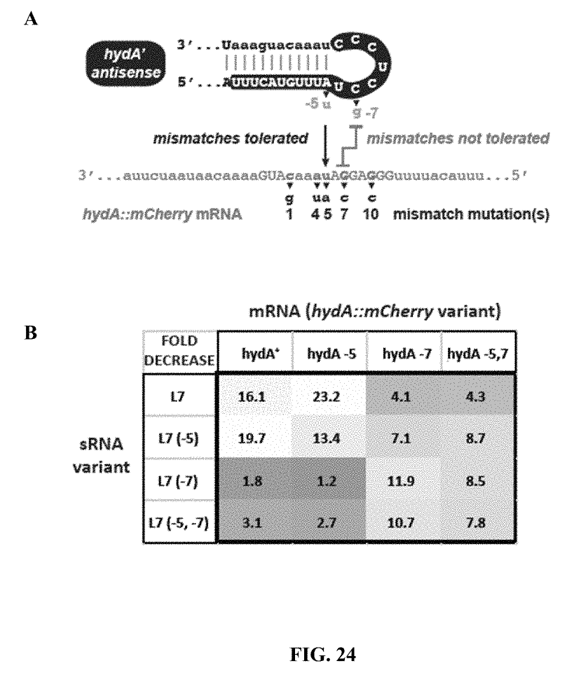

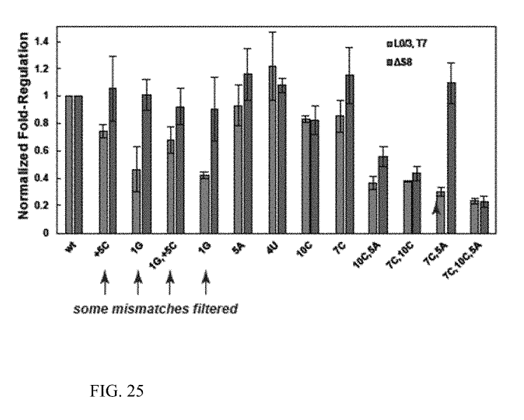

The efforts to create a more stable DsrA scaffold elucidated that the stem can act as a “sink” competitor for the mRNA target and filter out mismatches. To test this a series of hydA-mCherry reporter gene variants were created with mismatches in antisense region of TIR (i.e., positions predicted to base pair with specific structures in the fingerloop). The structured (stem-loop) and unstructured equivalent sRNAs were then compared. Filtering manifests as a strong difference in reporter gene response between structured and unstructured sRNAs. Unstructured (ΔS8) sRNA variant is active against mutant reporter gene constructs without much bias (FIG. 23, top) Fingerloop structured antisense filters mismatches in the loop, but not the stem, providing a basis of off-target mRNA filtering (FIG. 23, bottom). Results show that compensatory mutagenesis validates RNA:RNA interaction (FIG. 24). The behavior is consistent with both restoration of function in matching mutant pairs but also with loss of function by mutations on either strand that disrupt pairing in the fingerloop loop (FIG. 24). At the same time, toeholds were compared to determine if they enabled filtering. While filtering was enabled with toehold, the toehold T7 near the fingerloop does some off-target filtering, but the results were less robust than with loops (FIG. 25).

### Methods

Bacterial Strains, Plasmids and Growth Media.

E. coli K-12 strain DH5α (F⁻ endA1 glnV44 thi-1 recA1 relA1 gyrA96 deoR nupG purB20 φ80dlacZΔM15 Δ(lacZYA-argF) U169, hsdR17(r_{K}⁻m_{K}⁺), λ⁻) was used for plasmid construction and E. coli CM1000 (MG1655 ΔlacX74 dsrA14)(McCullen et al. 2010) was used for all gene expression analyses. The dsrA14 variant is a markerless null allele variant. Chemically competent cells prepared by the Inoue method were used for cloning sRNA variants and reporter genes. SOC medium was used for recovery of Inoue cells and Luria-Bertani lysogeny broth (LB) medium was used for transformations and recovery of frozen storage cultures using standard methods. (Sambrook and Russell 2001) Tryptone and yeast extract (DIFCO) were purchased from Fisher Scientific. Wild-type DsrA tuning experiments were performed in M9+GM (M9 medium)(Sambrook and Russell 2001) supplemented with Glucose (4 g/L) and trace metals (formulated per New Brunswick Scientific media). (Fong and Wood 2010) DsrA variants were analyzed in M9+GM plus 1% tryptone (M9+GMT). When required, appropriate inducers were added to the media: anhydrotetracycline (aTet, Sigma-Aldrich), L-arabinose (Ara, Acros Organics) and Isopropyl β-D-1-thiogalactopyranoside (IPTG, Fisher Scientific). For plasmid maintenance, ampicillin (Fisher Scientific), chloramphenicol (Acros Organics) and spectinomycin (MP Biomedicals) were added as appropriate to the media at 200, 25 and 75 μg/mL, respectively. For LB plates, 175 μg/mL of spectinomycin was added. Cells were grown at 37° C. for all experiments.

Details of plasmids, DNA oligonucleotides and restriction sites used can be found in Lahiry A, Stimple S D, Wood D W, Lease R A. Retargeting a Dual-Acting sRNA for Multiple mRNA Transcript Regulation. ACS Synthetic Biology. 2017; 6:648-58, which is expressly incorporated herein by reference. Briefly, the sRNA plasmid pSDS801a is based on pBR322 (amp^{R}, ori colE1), and encodes lacI P_{LlacO-1} dsr A P_{con} hfq. The GFPuv reporter plasmid is based on pACYC184 cat (cam^{R}, ori p15A) and encodes tetR Pₜₑₜ-gfpᵤᵥmut6. The mCherry reporter plasmid is based on pSC101 (spc^{R}, ori pSC101) and encodes araC P_{BAD}-mCherry P_{lac}lacY^{A177C}. Plasmids were constructed by standard DNA restriction and ligation cloning methods using either restriction-digested PCR products or annealed-oligonucleotide fragments, and confirmed by DNA sequencing.

Simultaneous Cell Growth and Fluorescence Assays.

For tests of the three-plasmid system (sRNA plasmid and two reporter plasmids), either fresh transformants or cells re-streaked from −80° C. glycerol storage cultures (Sambrook and Russell 2001) were grown for 16 h at 37° C. Single bacterial colonies were used to inoculate 2 mL M9+GM (or LB for retargeting experiments) for over-day cultures grown for 12 h, then diluted 1/100 (v/v) into individual wells of a 96-well Corning™ 3603 fluorescence microtiter plate with 150 μL of M9+GM medium and inducers (20 ng/mL aTet, 2% Ara, 1 mM IPTG), as appropriate. To suppress the evaporation of culture media during growth, 50 μl of mineral oil was overlaid onto each well. (Urban and Vogel 2007) Expression-tuning experiments followed this same protocol but with varying concentrations of inducers (0-20 ng/mL aTet, 0-2% Ara and 0-1 mM IPTG). The 96-well plates were grown shaking at 37° C. in a Biotek Synergy™ 2 Multi-Mode microplate reader in continuous readout mode for 12-16 h. Cell growth was measured as optical density at 600 nm (OD₆₀₀) and fluorescence measurements for GFPuvₘᵤₜ₆ (λₑₓ 395 nm, λₑₘ 509 nm maxima) and mCherry (λₑₓ 587 nm, λₑₘ 610 nm maxima) reporters were measured every 30 minutes. The plate reader settings were as follows for filters and dichroic mirrors: GFPuvₘᵤₜ₆ (excitation, 395±10 nm, emission, 528±10 with a 435 nm-cutoff dichroic mirror); mCherry (excitation, 585±5 nm, emission, 620±7.5 nm, with a 595 nm-cutoff dichroic mirror). Data from the plate reader were processed in an Excel spreadsheet by plotting fluorescence versus OD₆₀₀ and interpolating the fluorescence value at an OD₆₀₀ of 0.5 using a linear fit (R²>0.8), with heuristic analysis of linear fit data below this threshold in borderline cases. Background cellular auto-fluorescence was calculated using control strains containing the three empty plasmid vectors (pSDS1002, pACYCΔtetA and pBAD42) and was subtracted from the experimental fluorescence data. All reported plate assay data are the average of 3-7 biological sample replicates performed over at least 2 days. Error bars represent standard error of the mean. Rarely, experimental data measurements (3 total) were declared as outliers, defined as greater than 10 standard deviations from the mean, and were omitted from the analysis. Significance of sRNA-induced gene repression values was assessed via one-tailed matched pairs t-test (α=0.01).

Plasmid Construction.

The sRNA plasmids (FIG. 8A) were derived from pBR-plac-DsrA (Mandin and Gottesman 2010) and contained a ColE1 origin of replication and ampicillin resistance gene (bla). In this plasmid, dsrA is transcribed from an IPTG-inducible P_{LlacO-1} promoter. (Lutz and Bujard 1997; Mandin and Gottesman 2010) A series of restriction sites exist naturally within the dsrA gene, including ApoI (FIG. 2B). Because ApoI recognizes and cuts EcoRI sites, the internal ApoI site was rendered unique in plasmid pBR-plac-DsrA by alteration of the EcoRI site immediately downstream of dsrA. A constitutively-expressed lacI gene was added to improve repressor control (Glascock and Weickert 1998) with a downstream bi-directional transcript terminator. (Schollmeier et al. 1985) Due to interactions of DsrA and other sRNAs with the Sm-protein Hfq, (Fender et al. 2010; Hussein and Lim 2011; Sledjeski et al. 2001; Updegrove et al. 2016; Vogel and Luisi 2011) a copy of the Escherichia coli hfq gene was inserted into the sRNA plasmid, under the control of a constitutive promoter (Registry of Standard Biological parts: BBa_J23106) and a synthetic st3 ribosome binding site, (Vellanoweth and Rabinowitz 1992) with an E. coli rrnB T1 terminator downstream (Registry of Standard Biological parts: BBa_B0010). The DsrA plasmids were validated using a Miller assay (Miller 1972) of the strain M182 proU::lacZ, which increases beta-galactosidase activity when DsrA inhibits H-NS translation. (Gowrishankar 1985; Lease et al. 1998) All DsrA variants were cloned by annealing pairs of oligonucleotides to create fragments suitable for direct ligation with existing restriction sites (FIG. 2A).

The GFPuv reporter plasmids (FIG. 8B) were derived from pACYC184 (NEB), and contain a p15A origin of replication and chloramphenicol resistance (cat) gene. The GFPuvₘᵤₜ₆ allele (Timmes et al. 2004) was cloned by PCR under the control of a tetracycline-inducible promoter Pₜₑₜ with various target mRNA leaders fused in-frame with the GFPuvₘᵤₜ₆ sequence, and the tetR gene was cloned from the Tn10 transposon of the M182 proU::LacZ strain (Gowrishankar 1985; Lease et al. 1998) to provide inducible repressor control. Plasmid pUCBB-eGFP was a gift from Claudia Schmidt-Dannert (Addgene plasmid #32548). Plasmid pCyPet-His was a gift from Patrick Daugherty (Addgene plasmid #14030)

The mCherry reporter plasmids (FIG. 8C) were derived from pBAD42(Guzman et al. 1995) (Yale Coli Genetic Stock Center), and contain a pSC101 origin of replication and spectinomycin (spc^{R}) drug resistance gene. The pET-mCherry LIC cloning vector (u-mCherry) was a gift from Scott Gradia (Addgene plasmid #29769). The mCherry reporter gene was cloned under the control of the arabinose-inducible promoter P_{araBAD} with various target mRNA leaders fused in-frame with the mCherry sequence. The plasmid pLacY-A177C was a gift from the Cronan lab. (Morgan-Kiss et al. 2002) The lacY^{A177C} gene was inserted into the mCherry reporter plasmids under the control of a weak constitutive lacZ promoter (Registry of Standard Biological parts: BBa_K119000) for uniform dosage response from arabinose. High (2%) arabinose levels were required for full induction. Addition of the lacY^{A177C} gene and glucose in the medium improves uptake and minimizes degradation of arabinose as a carbon source, respectively; further optimization may require a knockout of the arabinose degradation genes. Others have demonstrated that sugar transporter proteins permit linear induction, (Khlebnikov et al. 2002) and in conjunction with arabinose catabolic mutants, diminish the requirement for high arabinose levels to achieve full induction. (Afroz et al. 2014)

Flow Cytometry.

The CM1000 strain containing appropriate reporter plasmids and the sRNA plasmid producing wild type DsrA were recovered from frozen storage cultures and grown overnight (16 h) on LB plates. A single colony was used to inoculate 2 mL M9+GM medium and grown for 12 hours. Cultures were diluted 1/200 in 5 mL M9+GM with appropriate inducers and cell growth was monitored by OD₅₉₅ using a spectrophotometer (Eppendorf BioPhotometer plus). At OD₅₉₅ 0.45-0.55, the cells were placed on ice (4° C. ice bath, 20 minutes) and recovered by centrifugation (5000×g, 2 min, RT) then resuspended in 500 μL of phosphate-buffered saline (MP Biomedicals PBS: 137 mM NaCl, 2.7 mM KCl, 8.1 mM Na₂HPO₄, and 1.47 mM KH₂PO₄, pH 7.4). Fluorescence measurements were performed by flow cytometry using a Becton-Dickinson FACSAria™ III machine equipped with 488 nm and 561 nm lasers and appropriate band pass filters to read GFPuvₘᵤₜ₆ (530/30) and mCherry (610/20) fluorescence.

### REFERENCES

Atsumi S, Cann A F, Connor M R, Shen C R, Smith K M, Brynildsen M P, Chou K J Y, Hanai T, Liao J C. 2008. Metabolic engineering of Escherichia coli for 1-butanol production. Metabolic Engineering 10(6):305-311.
Beisel C L, Storz G. 2011. The base-pairing RNA spot 42 participates in a multioutput feedforward loop to help enact catabolite repression in Escherichia coli. Mol Cell 41(3):286-97.
Beisel C L, Updegrove T B, Janson B J, Storz G. 2012. Multiple factors dictate target selection by Hfq-binding small RNAs. The EMBO Journal 31(8):1961-1974.
Bouloc P, Repoila F. 2016. Fresh layers of RNA-mediated regulation in Gram-positive bacteria. Current Opinion in Microbiology 30:30-35.
Brantl S, Bruckner R. 2014. Small regulatory RNAs from low-GC Gram-positive bacteria. RNA Biology 11(5):443-456.
Bruder M R, Pyne M E, Moo-Young M, Chung D A, Chou C P. 2016. Extending CRISPR-Cas9 Technology from Genome Editing to Transcriptional Engineering in the Genus Clostridium. Applied and Environmental Microbiology 82(20):6109-6119.
Caillet J, Gracia C, Fontaine F, Hajnsdorf E. 2014. Clostridium difficile Hfq can replace Escherichia coli Hfq for most of its function. Rna 20(10):1567-78.
Cayrol B, Fortas E, Martret C, Cech G, Kloska A, Caulet S, Barbet M, Trepout S, Marco S, Taghbalout A and others. 2015. Riboregulation of the bacterial actin-homolog MreB by DsrA small noncoding RNA. Integrative Biology 7(1):128-141.
Chen Y, Indurthi D C, Jones S W, Papoutsakis E T. 2011. Small RNAs in the Genus Clostridium. mBio 2(1):e00340-10.
Cho C, Lee S Y. 2016. Efficient gene knockdown in Clostridium acetobutylicum by synthetic small regulatory RNAs. Biotechnol Bioeng.
Cho S H, Haning K, Contreras L M. 2015. Strain engineering via regulatory noncoding RNAs: not a one-blueprint-fits-all. Current Opinion in Chemical Engineering 10:25-34.
de Almeida Ribeiro E, Beich-Frandsen M, Konarev P V, Shang W, Večrek B, Kontaxis G, Hämmerle H, Peterlik H, Svergun D I, Bläsi U and others. 2012. Structural flexibility of RNA as molecular basis for Hfq chaperone function. Nucleic Acids Research 40(16):8072-8084.
De Lay N, Schu D J, Gottesman S. 2013. Bacterial Small RNA-based Negative Regulation: Hfq and Its Accomplices. Journal of Biological Chemistry 288(12):7996-8003.
Fong B A, Wood D W. 2010. Expression and purification of ELP-intein-tagged target proteins in high cell density E. coli fermentation. Microb Cell Fact 9:77.
Girbal L, Soucaille P. 1994. Regulation of Clostridium acetobutylicum metabolism as revealed by mixed-substrate steady-state continuous cultures: role of NADH/NAD ratio and ATP pool. Journal of Bacteriology 176(21):6433-6438.
Gottesman S, Storz G. 2010. Bacterial Small RNA Regulators: Versatile Roles and Rapidly Evolving Variations. Cold Spring Harbor Perspectives in Biology.
Green A A, Silver P A, Collins J J, Yin P. 2014. Toehold switches: de-novo-designed regulators of gene expression. Cell 159(4):925-39.
Green E M, Boynton Z L, Harris L M, Rudolph F B, Papoutsakis E T, Bennett G N. 1996. Genetic manipulation of acid formation pathways by gene inactivation in Clostridium acetobutylicum ATCC 824. Microbiology 142(8):2079-2086.
Hao Y, Zhang Z J, Erickson D W, Huang M, Huang Y, Li J, Hwa T, Shi H. 2011. Quantifying the sequence-function relation in gene silencing by bacterial small RNAs. Proceedings of the National Academy of Sciences, USA 108(30):12473-12478.
Hussein R, Lim H N. 2011. Disruption of small RNA signaling caused by competition for Hfq. Proceedings of the National Academy of Sciences, USA 108(3):1110-1115.
Hussein R, Lim H N. 2012. Direct comparison of small RNA and transcription factor signaling. Nucleic Acids Res 40(15):7269-79.
Ishikawa H, Otaka H, Maki K, Morita T, Aiba H. 2012. The functional Hfq-binding module of bacterial sRNAs consists of a double or single hairpin preceded by a U-rich sequence and followed by a 3′ poly(U) tail. Rna 18(5):1062-74.
Jang Y-S, Lee J Y, Lee J, Park J H, Im J A, Eom M-H, Lee J, Lee S-H, Song H, Cho J-H and others. 2012. Enhanced Butanol Production Obtained by Reinforcing the Direct Butanol-Forming Route in Clostridium acetobutylicum. mBio 3(5): e00314-12.
Kang Z, Wang X, Li Y, Wang Q, Qi Q. 2012. Small RNA RyhB as a potential tool used for metabolic engineering in Escherichia coli. Biotechnol Lett 34(3):527-31.
Keasling J D. 2008. Synthetic Biology for Synthetic Chemistry. ACS Chemical Biology 3(1):64-76.
Knoshaug E P, Zhang M. 2009. Butanol tolerance in a selection of microorganisms. Appl Biochem Biotechnol 153(1-3):13-20.
Kushwaha M, Rostain W, Prakash S, Duncan J N, Jaramillo A. 2016. Using RNA as Molecular Code for Programming Cellular Function. ACS Synth Biol 5(8):795-809.
Lalaouna D, Masse E. 2016. The spectrum of activity of the small RNA DsrA: not so narrow after all. Curr Genet 62(2):261-4.
Lalaouna D, Morissette A, Carrier M-C, Massé E. 2015. DsrA regulatory RNA represses both has and rbsD mRNAs through distinct mechanisms in Escherichia coli. Molecular Microbiology 98(2):357-369.
Lease R A, Belfort M. 2000. A trans-acting RNA as a control switch in Escherichia coli: DsrA modulates function by forming alternative structures. Proc Natl Acad Sci USA 97(18):9919-24.
Lease R A, Cusick M E, Belfort M. 1998. Riboregulation in Escherichia coli: DsrA RNA acts by RNA:RNA interactions at multiple loci. Proc Natl Acad Sci USA 95(21):12456-61.
Lease R A, Smith D, McDonough K, Belfort M. 2004. The small noncoding DsrA RNA is an acid resistance regulator in Escherichia coli. J Bacteriol 186(18):6179-85.
Lease R A, Woodson S A. 2004. Cycling of the Sm-like protein Hfq on the DsrA small regulatory RNA. J Mol Biol 344(5):1211-23.
Levine E, Zhang Z, Kuhlman T, Hwa T. 2007. Quantitative Characteristics of Gene Regulation by Small RNA. PLoS Biol 5(9):e229.
Li F, Wang Y, Gong K, Wang Q, Liang Q, Qi Q. 2014. Constitutive expression of RyhB regulates the heme biosynthesis pathway and increases the 5-aminolevulinic acid accumulation in Escherichia coli. FEMS Microbiol Lett 350(2):209-15.
Li Q, Chen J, Minton N P, Zhang Y, Wen Z, Liu J, Yang H, Zeng Z, Ren X, Yang J and others. 2016. CRISPR-based genome editing and expression control systems in Clostridium acetobutylicum and Clostridium beijerinckii. Biotechnology Journal 11(7):961-972.
Liu R, Bassalo M C, Zeitoun R I, Gill R T. 2015. Genome scale engineering techniques for metabolic engineering. Metabolic Engineering 32:143-154.
Liu Y, Zhu Y, Li J, Shin H D, Chen R R, Du G, Liu L, Chen J. 2014. Modular pathway engineering of Bacillus subtilis for improved N-acetylglucosamine production. Metab Eng 23:42-52.
Majdalani N, Cunning C, Sledjeski D, Elliott T, Gottesman S. 1998. DsrA RNA regulates translation of RpoS message by an anti-antisense mechanism, independent of its action as an antisilencer of transcription. Proc Natl Acad Sci USA 95(21):12462-7.
Mandin P, Gottesman S. 2010. Integrating anaerobic/aerobic sensing and the general stress response through the ArcZ small RNA. EMBO J 29(18):3094-107.
Masse E, Escorcia F E, Gottesman S. 2003. Coupled degradation of a small regulatory RNA and its mRNA targets in Escherichia coli. Genes Dev 17(19):2374-83.
McCullen C A, Benhammou J N, Majdalani N, Gottesman S. 2010. Mechanism of Positive Regulation by DsrA and RprA Small Noncoding RNAs: Pairing Increases Translation and Protects rpoS mRNA from Degradation. J Bacteriol 192(21):5559-5571.
Mitarai N, Benjamin J A, Krishna S, Semsey S, Csiszovszki Z, Masse E, Sneppen K. 2009. Dynamic features of gene expression control by small regulatory RNAs. Proc Natl Acad Sci USA 106(26):10655-9.
Na D, Yoo S M, Chung H, Park H, Park J H, Lee S Y. 2013. Metabolic engineering of Escherichia coli using synthetic small regulatory RNAs. Nat Biotechnol 31(2):170-4.
Nakayama S-i, Kosaka T, Hirakawa H, Matsuura K, Yoshino S, Furukawa K. 2008. Metabolic engineering for solvent productivity by downregulation of the hydrogenase gene cluster hupCBA in Clostridium saccharoperbutylacetonicum strain N1-4. Applied Microbiology and Biotechnology 78(3):483-493.
Nielsen L K. 2011. Metabolic engineering: From retrofitting to green field. Nat Chem Biol 7(7):408-409.
Panja S, Santiago-Frangos A, Schu D J, Gottesman S, Woodson S A. 2015. Acidic Residues in the Hfq Chaperone Increase the Selectivity of sRNA Binding and Annealing. Journal of Molecular Biology 427(22):3491-3500.
Park H, Bak G, Kim S C, Lee Y. 2013. Exploring sRNA-mediated gene silencing mechanisms using artificial small RNAs derived from a natural RNA scaffold in Escherichia coli. Nucleic Acids Res 41(6):3787-804.
Peer A, Margalit H. 2011. Accessibility and Evolutionary Conservation Mark Bacterial Small-RNA Target-Binding Regions. Journal of Bacteriology 193(7):1690-1701.
Peters G, Coussement P, Maertens J, Lammertyn J, De Mey M. 2015. Putting RNA to work: Translating RNA fundamentals into biotechnological engineering practice. Biotechnol Adv 33(8):1829-44.
Sakai Y, Abe K, Nakashima S, Yoshida W, Ferri S, Sode K, Ikebukuro K. 2014. Improving the Gene-Regulation Ability of Small RNAs by Scaffold Engineering in Escherichia coli. ACS Synthetic Biology 3(3):152-162.
Sambrook J, Russell D W. 2001. Molecular Cloning: A Laboratory Manual (3rd ed.): Cold Spring Harbor Laboratory Press, Cold Spring Harbor, N.Y.
Schmiedel J M, Axmann I M, Legewie S. 2012. Multi-Target Regulation by Small RNAs Synchronizes Gene Expression Thresholds and May Enhance Ultrasensitive Behavior. PLoS ONE 7(8):e42296.
Schu D J, Zhang A, Gottesman S, Storz G. 2015. Alternative Hfq-sRNA interaction modes dictate alternative mRNA recognition. Embo j 34(20):2557-73.
Sharma V, Yamamura A, Yokobayashi Y. 2012. Engineering Artificial Small RNAs for Conditional Gene Silencing in Escherichia coli. ACS Synthetic Biology 1(1):6-13.
Sledjeski D D, Gupta A, Gottesman S. 1996. The small RNA, DsrA, is essential for the low temperature expression of RpoS during exponential growth in Escherichia coli. Embo J 15(15):3993-4000.
Storz G, Vogel J, Wassarman K M. 2011. Regulation by small RNAs in bacteria: expanding frontiers. Mol Cell 43(6):880-91.
Tashiro Y, Shinto H, Hayashi M, Baba S-i, Kobayashi G, Sonomoto K. 2007. Novel high-efficient butanol production from butyrate by non-growing Clostridium saccharoperbutylacetonicum N1-4 (ATCC 13564) with methyl viologen. Journal of Bioscience and Bioengineering 104(3):238-240.
Tummala S B, Junne S G, Papoutsakis E T. 2003. Antisense RNA Downregulation of Coenzyme A Transferase Combined with Alcohol-Aldehyde Dehydrogenase Overexpression Leads to Predominantly Alcohologenic Clostridium acetobutylicum Fermentations. J. Bacteriol. 185(12):3644-3653.
Updegrove T B, Zhang A, Storz G. 2016. Hfq: the flexible RNA matchmaker. Current Opinion in Microbiology 30:133-138.
Urban J H, Vogel J. 2007. Translational control and target recognition by Escherichia coli small RNAs in vivo. Nucleic Acids Res 35(3):1018-37.
Vazquez-Anderson J, Contreras L M. 2013. Regulatory RNAs: Charming gene management styles for synthetic biology applications. RNA Biology 10(12):1778-1797.
Venkataramanan K, Jones S, McCormick K, Kunjeti S, Ralston M, Meyers B, Papoutsakis E. 2013. The Clostridium small RNome that responds to stress: the paradigm and importance of toxic metabolite stress in C. acetobutylicum. BMC Genomics 14(1):849.
Wagner E G, Romby P. 2015. Small RNAs in bacteria and archaea: who they are, what they do, and how they do it. Adv Genet 90:133-208.
Wang J S, Zhang D Y. 2015. Simulation-guided DNA probe design for consistently ultraspecific hybridization. Nat Chem 7(7):545-553.
Yu M, Zhang Y, Tang I C, Yang S-T. 2011. Metabolic engineering of Clostridium tyrobutyricum for n-butanol production. Metabolic Engineering 13(4):373-382.
Zadeh J N, Steenberg C D, Bois J S, Wolfe B R, Pierce M B, Khan A R, Dirks R M, Pierce N A. 2011. NUPACK: Analysis and design of nucleic acid systems. J Comput Chem 32(1):170-3.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

We claim:

1. A system for measuring the activity of a chimeric nucleic acid in a cell, comprising:
a first plasmid comprising a chimeric nucleic acid, wherein the chimeric nucleic acid comprises a first nucleic acid sequence operably linked to a second nucleic acid sequence;
a second plasmid comprising a first reporter gene operably linked to a first gene leader sequence; and
a third plasmid comprising a second reporter gene operably linked to a second gene leader sequence;
wherein the first nucleic acid sequence is present in a first fingerloop stem loop and the second nucleic acid sequence is present in a second fingerloop stem loop; and
wherein the first and second fingerloop stem loops inhibit the binding of the first and second nucleic acids to mismatched target sequences.

2. The system of claim 1, wherein the first nucleic acid sequence and the second nucleic acid sequence encode an sRNA comprised in at least two fingerloop structures.

3. The system of claim 1, wherein the chimeric nucleic acid encodes for a chimeric small regulatory RNA.

4. The system of claim 1, wherein the first and second nucleic acids are positioned in antisense fingerloop regions of a gene encoding an endogenous small regulatory RNA of the cell.

5. The system of claim 1, wherein the first plasmid comprises an inducible promoter operably linked to the chimeric nucleic acid.

6. The system of claim 1, wherein an sRNA of the first nucleic acid sequence binds to an mRNA of the first gene leader sequence.

7. The system of claim 1, wherein an sRNA of the second nucleic acid sequence binds to an mRNA of the second gene leader sequence.

8. The system of claim 1, wherein the first reporter gene or the second reporter gene encodes a fluorescent protein.

9. The system of claim 1, wherein the chimeric nucleic acid is from about 50 to about 300 nucleotides in length.

10. The system of claim 1, wherein the first and second gene leader sequences target genes in the same metabolic pathway.

11. A method for modulating protein expression levels or mRNA expression levels from at least two target mRNAs in a cell simultaneously, the method comprising:
transforming the cell with a system for measuring the activity of a chimeric nucleic acid, the system comprising:
a first plasmid comprising a chimeric nucleic acid, wherein the chimeric nucleic acid comprises a first nucleic acid sequence operably linked to a second nucleic acid sequence;
a second plasmid comprising a first reporter gene operably linked to a first gene leader sequence; and
a third plasmid comprising a second reporter gene operably linked to a second gene leader sequence;
wherein the first nucleic acid sequence is present in a first fingerloop stem loop and the second nucleic acid sequence is present in a second fingerloop stem loop;
wherein the first and second fingerloop stem loops inhibit the binding of the first and second nucleic acids to mismatched target sequences;
wherein an sRNA of the first nucleic acid sequence binds to an mRNA of the first gene leader sequence and an sRNA of the second nucleic acid sequence binds to an mRNA of the second gene leader sequence; and
measuring the protein expression levels or mRNA expression levels of the first reporter gene and the second reporter gene.

12. The method of claim 11, wherein the first nucleic acid sequence and the second nucleic acid sequence encode an sRNA comprised in at least two fingerloop structures.

13. The method of claim 11, wherein the chimeric nucleic acid encodes for a chimeric small regulatory RNA.

14. The method of claim 11, wherein the first reporter gene or the second reporter gene encodes a fluorescent protein.

15. The method of claim 11, wherein the chimeric nucleic acid is from about 50 to about 300 nucleotides in length.

16. The method of claim 11, wherein the cell is an Escherichia coli cell.

17. The method of claim 11, wherein the cell is an Clostridium acetobutylicum cell.

18. The method of claim 11, wherein the chimeric nucleic acid binds to the at least two target mRNAs encoding at least two cell enzymes, and wherein binding results in a reduction of activity of the at least two cell enzymes.

19. The method of claim 11, wherein the at least two target mRNAs are in the same metabolic pathway.

20. The method of claim 11, wherein the at least two target mRNAs are in different metabolic pathways.
